(19) United States
(12) Patent Application Publication
(10) Pub. No.: US 2014/0176602 A1
(43) Pub. Date: Jun. 26, 2014

(54) INFORMATION PROCESSING SYSTEM, INFORMATION PROCESSING METHOD, INFORMATION PROCESSING APPARATUS, AND CONTROL METHOD AND CONTROL PROGRAM THEREOF

(71) Applicant: NEC CORPORATION, Tokyo (JP)

(72) Inventors: Yoshiko Yoshihara, Tokyo (JP); Tomoharu Kiyuna, Tokyo (JP); Toru Sano, Tokyo (JP); Kenichi Kamjo, Tokyo (JP)

(73) Assignee: NEC CORPORATION, Tokyo (JP)

(21) Appl. No.: 14/239,076

(22) PCT Filed: Aug. 17, 2012
(86) PCT No.: PCT/JP2012/005182

§ 371 Date: Feb. 14, 2014

(30) Foreign Application Priority Data
Aug. 18, 2011 (JP) 2011-179094

(51) Int. Cl. A61B 5/00; G06T 7/00; G06T 11/60

(52) U.S. Cl. CPC A61B 5/0033; G06T 11/60; G06T 7/0014
USPC 345/629

(57) ABSTRACT

Provided is an information processing apparatus which supports diagnosis based on a tissue specimen image obtained by capturing body tissues, including an area generation unit that divides at least one feature value of the tissue specimen image into a plurality of levels based on a magnitude of the feature value, and generates an area on the tissue specimen image belonging to each level; and an overlay image generation unit that associates the area of each level which is generated by the area generation unit with an image which is processed to have the same shape and the same positional relation as the area and in which a magnitude relation of the feature value is identifiable and generates an overlay image.

### TECHNICAL FIELD

The present invention relates to an information processing technology for supporting diagnosis based on tissue specimen images obtained by capturing body tissues.

### BACKGROUND ART

In the field of the technology described above, as disclosed in Patent Document 1, a technology for color coding and displaying an angle in the longitudinal direction of a nucleus of a signet-ring cell so as to facilitate determination of a shape of the nucleus of the signet-ring cell has been known. Further, Patent Document 2 discloses a technology for dividing a tissue specimen image into grid-like areas so as to determine and display the importance of each divided area.

### RELATED DOCUMENT

### Patent Document

[Patent Document 1] Japanese Unexamined Patent Publication No. 2009-180539
[Patent Document 2] Japanese Unexamined Patent Publication No. 2010-281637

### DISCLOSURE OF THE INVENTION

However, in the technologies described in the above Patent Documents, a pathologist cannot determine at a glance at which level and in which range the feature values for pathological diagnosis are distributed, while observing tissue specimen images.

An object of the present invention is to provide a technology for solving the problems described above.

In order to achieve the object, according to the present invention, there is provided an information processing apparatus which supports diagnosis based on a tissue specimen image obtained by capturing body tissues, and includes an area generation unit that divides at least one feature value of the tissue specimen image into a plurality of levels based on the magnitude of the feature value, and generates an area on the tissue specimen image belonging to each level; and an overlay image generation unit that associates the area of each level which is generated by the area generation unit with an image which is processed to have the same shape and the same positional relation as the area and in which a magnitude relation of the feature value is identifiable, and generates an overlay image.

In order to achieve the object, according to the present invention, there is provided a control method of an information processing apparatus which supports diagnosis based on a tissue specimen image obtained by capturing body tissues, and includes an area generation step of dividing at least one feature value of the tissue specimen image into a plurality of levels based on the magnitude of the feature value, and generates an area on the tissue specimen image belonging to each level; and an overlay image generation step of associating the area of each level which is generated in the area generation step with an image which is processed to have the same shape and the same positional relation as the area and in which a magnitude relation of the feature value is identifiable, and generates an overlay image.

In order to achieve the object, according to the present invention, there is provided a control program of an information processing apparatus which supports diagnosis based on a tissue specimen image obtained by capturing body tissues, and causes a computer to implement an area generation step of dividing at least one feature value of the tissue specimen image into a plurality of levels based on the magnitude of the feature value, and generates an area on the tissue specimen image belonging to each level; and an overlay image generation step of associating the area of each level which is generated in the area generation step with an image which is processed to have the same shape and the same positional relation as the area and in which a magnitude relation of the feature value is identifiable, and generates an overlay image.

In order to achieve the object, according to the present invention, there is provided an information processing system which supports diagnosis based on a tissue specimen image obtained by capturing body tissues, and includes an input unit that inputs the captured tissue specimen image; an area generation unit that divides at least one feature value of the tissue specimen image into a plurality of levels based on the magnitude of the feature value, and generates an area on the tissue specimen image belonging to each level; an overlay image generation unit that associates the area of each level which is generated by the area generation unit with an image which is processed to have the same shape and the same positional relation as the area and in which a magnitude relation of the feature value is identifiable, and generates an overlay image; and a superimposing and displaying unit that superimposes the overlay image generated by the overlay image generation unit on the tissue specimen image and displays the superimposed image.

In order to achieve the object, according to the present invention, there is provided an information processing method which supports diagnosis based on a tissue specimen image obtained by capturing body tissues, and includes an input step of inputting the captured tissue specimen image; an area generation step of dividing at least one feature value of the tissue specimen image into a plurality of levels based on the magnitude of the feature value, and generating an area on the tissue specimen image belonging to each level; an overlay image generation step of associating the area of each level which is generated in the area generation step with an image which is processed to have the same shape and the same positional relation as the area and in which a magnitude relation of the feature value is identifiable and generating an overlay image; and a superimposing and displaying step of superimposing the overlay image generated in the overlay image generation step on the tissue specimen image and displaying the superimposed image.

According to the present exemplary embodiment, a pathologist can determine at a glance at which level and in which range the feature values for pathological diagnosis are distributed, while observing tissue specimen images.

### BRIEF DESCRIPTION OF THE DRAWINGS

Above described object and other objects, features and advantages will become more apparent by preferred exemplary embodiments described below and the following accompanying drawings.

FIG. 1 is a block diagram showing a configuration of an information processing apparatus according to a first exemplary embodiment of the present invention.

FIG. 2 is a block diagram showing a configuration of an information processing apparatus according to a second exemplary embodiment of the present invention.

FIG. 3A is a diagram showing a display of a tissue specimen image according to the second exemplary embodiment of the present invention.

FIG. 3B is a diagram showing a display of an overlay image according to the second exemplary embodiment of the present invention.

FIG. 3C is a diagram showing a display of a tissue specimen image on which the overlay image according to the second exemplary embodiment of the present invention is superimposed.

FIG. 3D is a diagram showing displays of the tissue specimen image, and a tissue specimen image on which the overlay image is superimposed according to the second exemplary embodiment of the present invention.

FIG. 4A is a diagram showing a configuration example of a feature value DB according to the second exemplary embodiment of the present invention.

FIG. 4B is a diagram showing a configuration example of the feature value DB according to the second exemplary embodiment of the present invention.

FIG. 4C is a diagram showing a configuration example of the feature value DB according to the second exemplary embodiment of the present invention.

FIG. 4D is a diagram showing a configuration example of the feature value DB according to the second exemplary embodiment of the present invention.

FIG. 4E is a diagram showing a configuration example of the feature value DB according to the second exemplary embodiment of the present invention.

FIG. 5 is a diagram showing a configuration of a level classification DB according to the second exemplary embodiment of the present invention.

FIG. 6 is a diagram showing a configuration of an allocation image DB according to the second exemplary embodiment of the present invention.

FIG. 7A is a diagram showing an example of area information according to the second exemplary embodiment of the present invention.

FIG. 7B is a diagram showing another example of the area information according to the second exemplary embodiment of the present invention.

FIG. 7C is a diagram showing still another example of the area information according to the second exemplary embodiment of the present invention.

FIG. 8A is a diagram showing an example of overlay image information according to the second exemplary embodiment of the present invention.

FIG. 8B is a diagram showing another example of the overlay image information according to the second exemplary embodiment of the present invention.

FIG. 9 is a block diagram showing a hardware configuration of the information processing apparatus according to the second exemplary embodiment of the present invention.

FIG. 10 is a flowchart showing a processing procedure of the information processing apparatus according to the second exemplary embodiment of the present invention.

FIG. 11 is a block diagram showing a configuration of an information processing system according to a third exemplary embodiment of the present invention.

FIG. 12 is a diagram showing a configuration of an allocation image DB according to the third exemplary embodiment of the present invention.

FIG. 13 is a diagram showing overlay image information according to the third exemplary embodiment of the present invention.

FIG. 14 is a block diagram showing a configuration of an information processing system according to a fourth exemplary embodiment of the present invention.

FIG. 15 is a diagram showing a screen for selecting a feature value and a level image according to the fourth exemplary embodiment of the present invention.

FIG. 16 is a sequence diagram showing an operation procedure of the information processing system according to the fourth exemplary embodiment of the present invention.

FIG. 17 is a block diagram showing a hardware configuration of the information processing apparatus according to the fourth exemplary embodiment of the present invention.

FIG. 18 is a flowchart showing a processing procedure of the information processing apparatus according to the fourth exemplary embodiment of the present invention.

FIG. 19 is a block diagram showing a configuration of an information processing system according to a fifth exemplary embodiment of the present invention.

FIG. 20 is a diagram showing a screen for specifying a tissue specimen image according to the fifth exemplary embodiment of the present invention.

FIG. 21 is a diagram showing a configuration of a table for determination according to the fifth exemplary embodiment of the present invention.

FIG. 22 is a flowchart showing a processing procedure of the information processing apparatus according to the fifth exemplary embodiment of the present invention.

FIG. 23 is a block diagram showing a configuration of an information processing system according to a sixth exemplary embodiment of the present invention.

FIG. 24 is a drawing showing a screen for expanding an area of a tissue specimen image according to the sixth exemplary embodiment of the present invention.

FIG. 25 is a diagram showing a configuration of a magnification selection table according to the sixth exemplary embodiment of the present invention.

FIG. 26 is a diagram showing a configuration of expanding transmission data according to the sixth exemplary embodiment of the present invention.

FIG. 27 is a flowchart showing a processing procedure of the information processing apparatus according to the sixth exemplary embodiment of the present invention.

FIG. 28 is a drawing showing a screen in which an overlay image is superimposed on a plurality of tissue specimen images according to a seventh exemplary embodiment of the present invention.

FIG. 29 is a drawing showing area information according to the seventh exemplary embodiment of the present invention.

FIG. 30 is a drawing showing overlay image information according to the seventh exemplary embodiment of the present invention.

### DESCRIPTION OF EXEMPLARY EMBODIMENTS

Hereinafter, exemplary embodiments of the present invention will be described in detail byway of examples with reference to drawings. However, constituent elements described in the following exemplary embodiments are merely illustrative, and the technical scope of the present invention is not intended to be limited only thereto.

### First Exemplary Embodiment

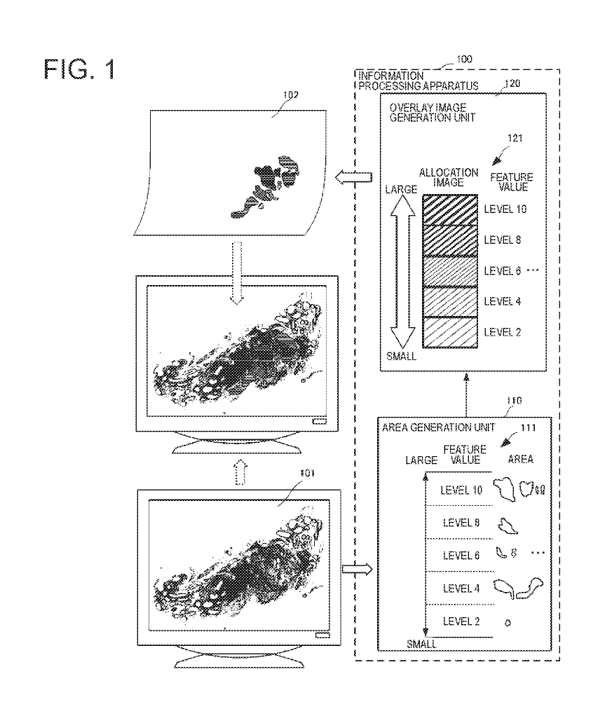

An information processing apparatus 100 as a first exemplary embodiment of the present invention will be described using FIG. 1. The information processing apparatus 100 is an apparatus which supports diagnosis based on a tissue specimen image obtained by capturing body tissues.

As shown in FIG. 1, the information processing apparatus 100 includes an area generation unit 110 and an overlay image generation unit 120. The area generation unit 110 divides at least one feature value of a tissue specimen image 101 into a plurality of levels based on a magnitude of the feature value, and generates an area 111 on the tissue specimen image belonging to each level. The overlay image generation unit 120 associates the area 111 of each level which is generated by the area generation unit 110 with an image 121 which is processed to have the same shape and the same positional relation as the area and in which a magnitude relation of the feature value is identifiable and generates an overlay image 102.

According to the present exemplary embodiment, a pathologist can determine at a glance at which level and in which range the feature values for pathological diagnosis are distributed, while observing tissue specimen images.

### Second Exemplary Embodiment

Next, an information processing system according to a second exemplary embodiment of the present invention will be described. In the present exemplary embodiment, the information processing apparatus 100 sets areas depending on feature values or the levels of the feature values within the tissue specimen images to be diagnosed by the pathologist, and associates each area with an image which is processed to have the same shape and the same positional relation as the area and in which the level of the feature value is identifiable by colors and patterns. Then, the information processing apparatus 100 generates an overlay image including an allocation image and transmits the overlay image to the communication terminal of the pathologist. The communication terminal of the pathologist superimposes the overlay image on the tissue specimen image and displays the superimposed image.

The present exemplary embodiment enables a support for facilitating the transition to the subsequent operation by the pathologist of the tissue sample image to be diagnosed, for example, the selection of an attention area and the expansion of an area to be diagnosed in detail.

### <<Configuration of Information Processing System>>

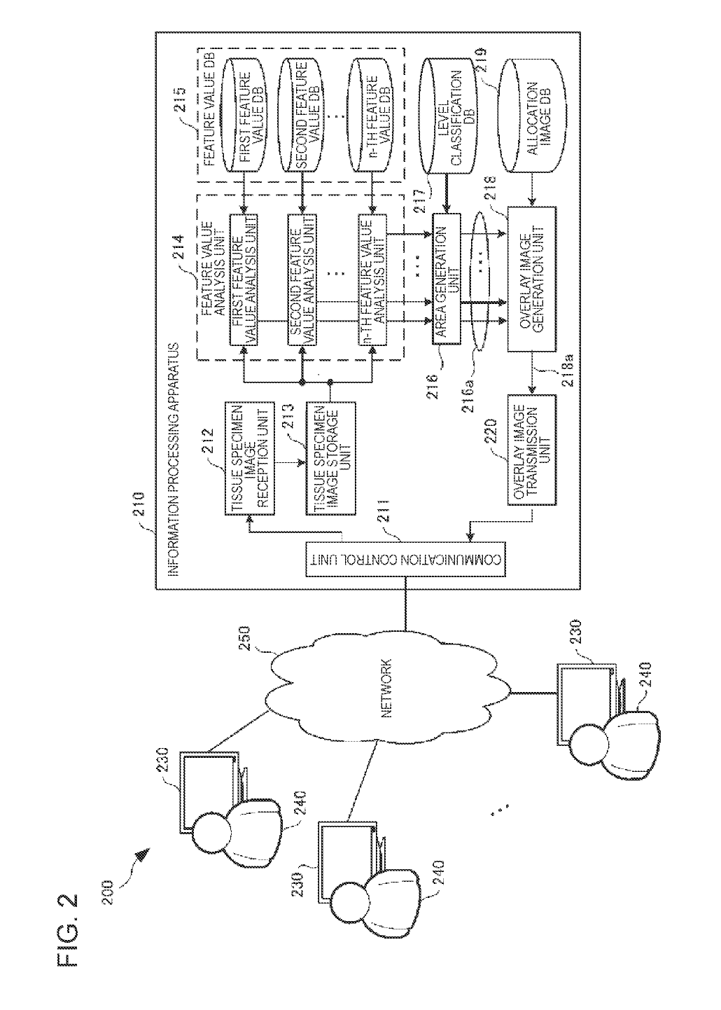

FIG. 2 is a block diagram showing a configuration of an information processing system 200 according to the second exemplary embodiment of the present invention.

The information processing system 200 includes an information processing apparatus 210 which is a pathological diagnosis support apparatus and is connected through a network 250, and communication terminals 230 which is operable by pathologists 240 and receive pathological diagnosis supports. In addition, the network 250 may be LANs in hospitals, or public lines or wireless communication connected with the outside of the hospitals.

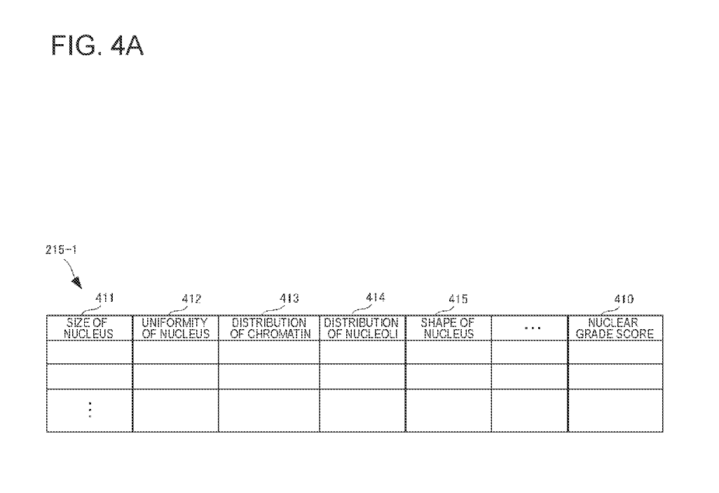

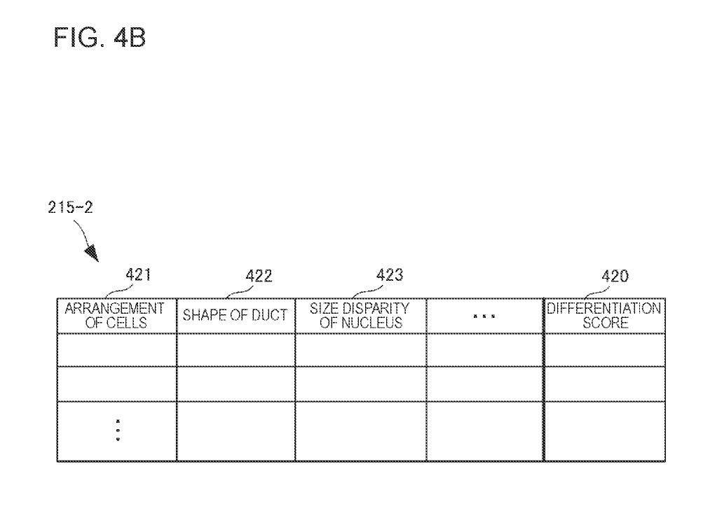

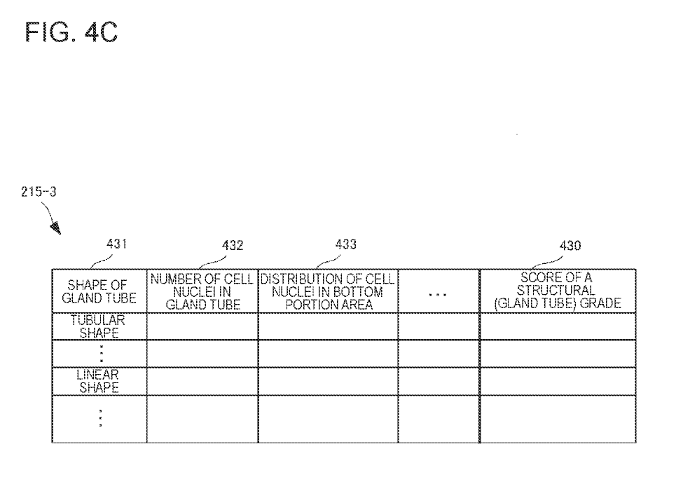

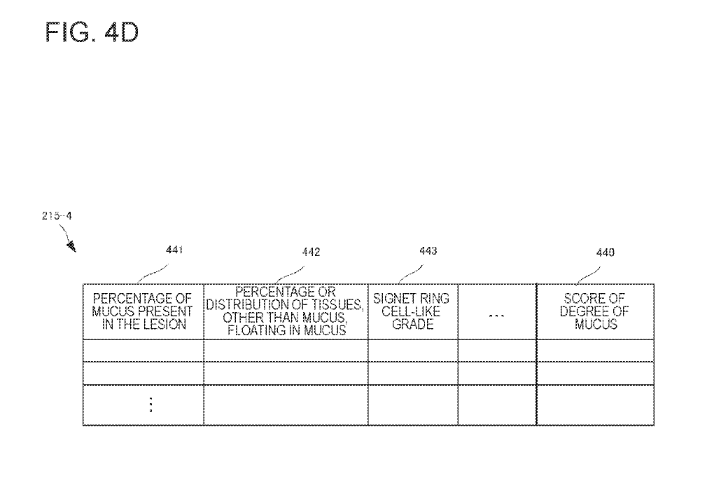

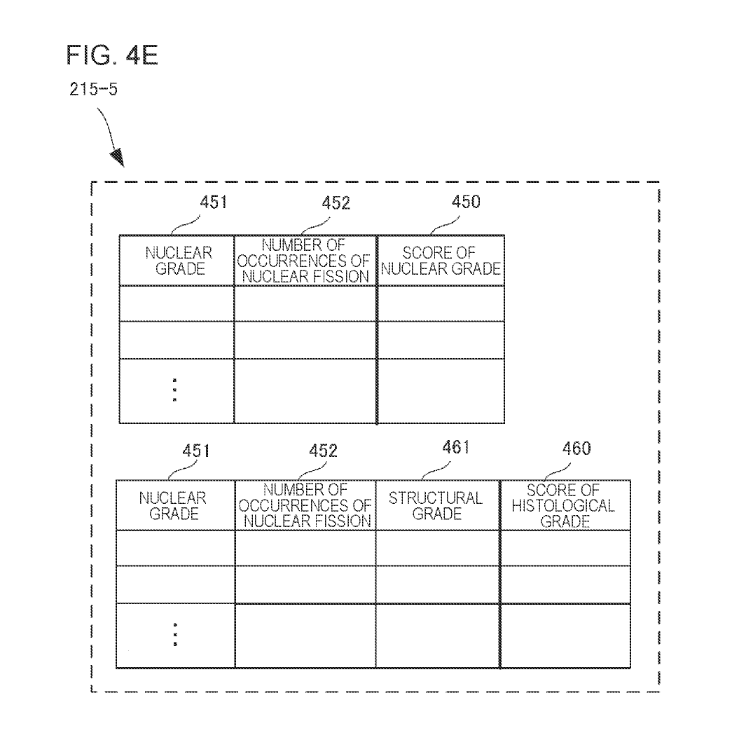

The information processing apparatus 210 includes a communication control unit 211 that controls communication with the communication terminals 230 through the network 250. Through the communication control unit 211, the tissue specimen images received from communication terminals 230 by a tissue specimen image reception unit 212 are stored in a tissue specimen image storage unit 213. Then, the information processing apparatus 210 obtains the feature values of the stored tissue specimen images by referring to information of a feature value database 215 (hereinafter, referred to as DBs: see FIG. 4A to FIG. 4E) corresponding to a feature value analysis unit 214.

The feature value may be one or plural as in FIG. 2. For example, the feature value includes a degree of differentiation representing the degree of differentiation of cancer cells, a grade that is an evaluation of a histopathological grade of cancer cells, a nuclear grade which is an evaluation by the size or the shape of a cell nucleus, a structural grade representing a degree of gland tube formation, a number or a percentage of occurrences of nuclear fission of the cell nucleus, a degree of mucus secreted from the mucosa and the gland, and a possibility of a signet-ring cell cancer. Further, it is possible to use any combination of the feature values, as a feature value.

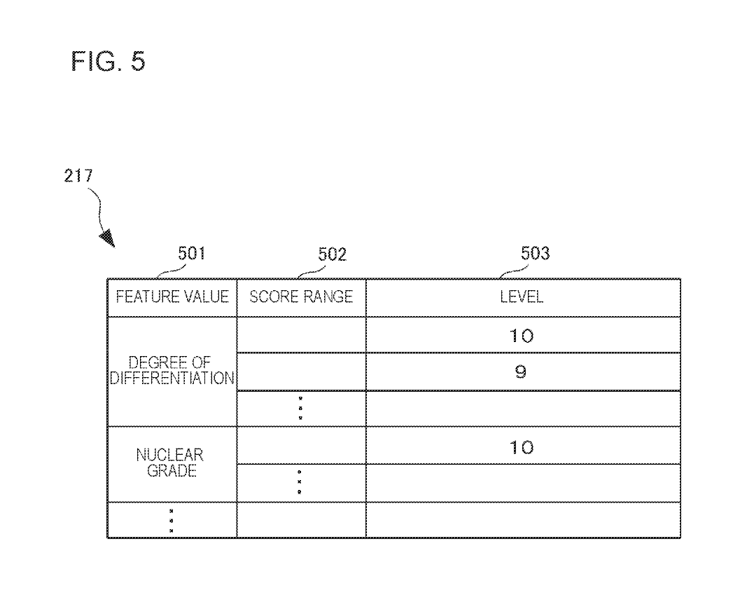

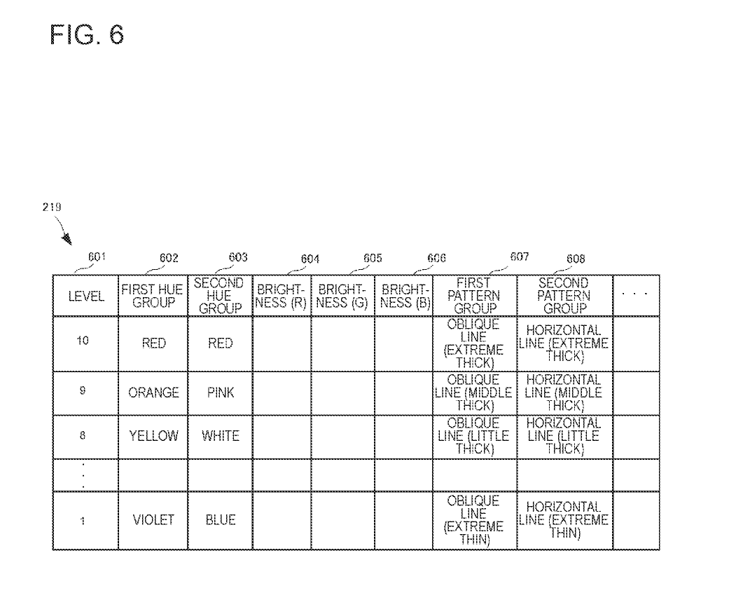

An area generation unit 216 divides, with reference to a level classification DB 217 (see FIG. 5), the feature value received from the feature value analysis unit 214 into a plurality of levels, and generates area information 216a having a common level. At this time, the area generation unit 216 generates each area, while maintaining the relative positional relation on the tissue specimen image which is a basis. An overlay image generation unit 218 processes the image (identifiable by colors and patterns) which is allocated corresponding to the feature value or the level, which are stored in allocation image DB 219 (see FIG. 6), to have the same shape and the same positional relation as the area, and associates the processed image with each area. At this time, the overlay image generation unit 218 associates each area with the image, using the relative positional relation maintained by the area generation unit 216. Then, the overlay image generation unit 218 generates overlay image information 218a including the area associated with the image. An overlay image transmission unit 220 transmits the overlay image information 218a to the communication terminal 230 of the pathologist 240 through the network 250 by the communication control unit 211.

The communication terminal 230 superimposes the tissue specimen image that has been transmitted to the information processing apparatus 210 on the received overlay image and displays the superimposed image. Here, since the overlay image is generated while maintaining relative positional relations among a plurality of areas as described above, the overlay image is coincident with the tissue specimen image which is a base for generating the area in the positional relation. Therefore, when the images are superimposed, the communication terminal 230 can align the positional relation thereof. In addition, although only the overlay image is transmitted in the present exemplary embodiment, the information processing apparatus 210 may transmit a superimposed image in which the tissue specimen image is superimposed on the overlay image. However, it is desirable to transmit only the overlay image in view of communication traffic.

### (Display Screen)

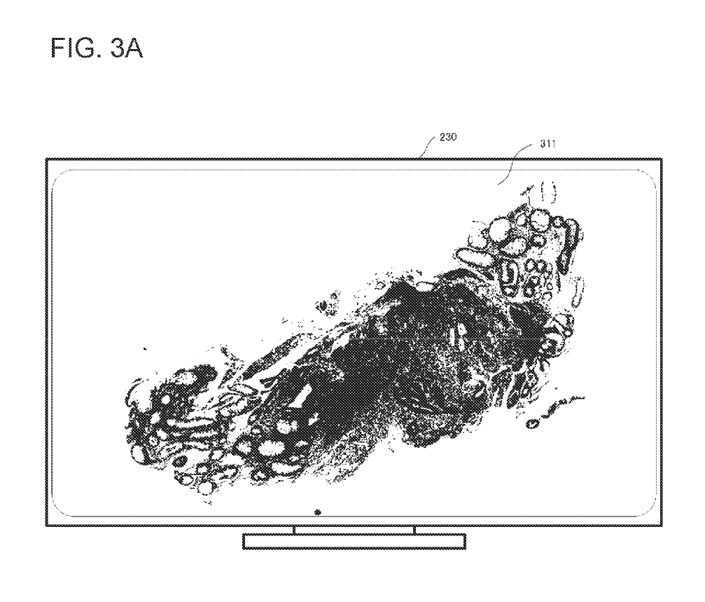

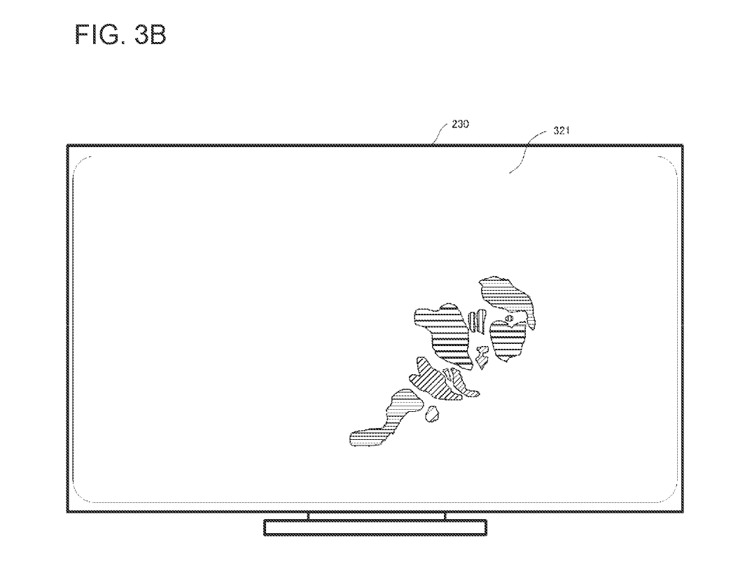

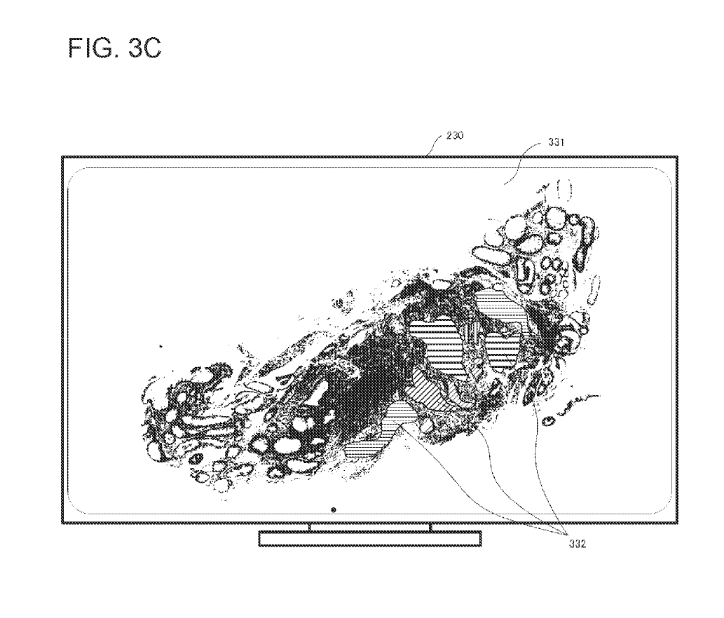

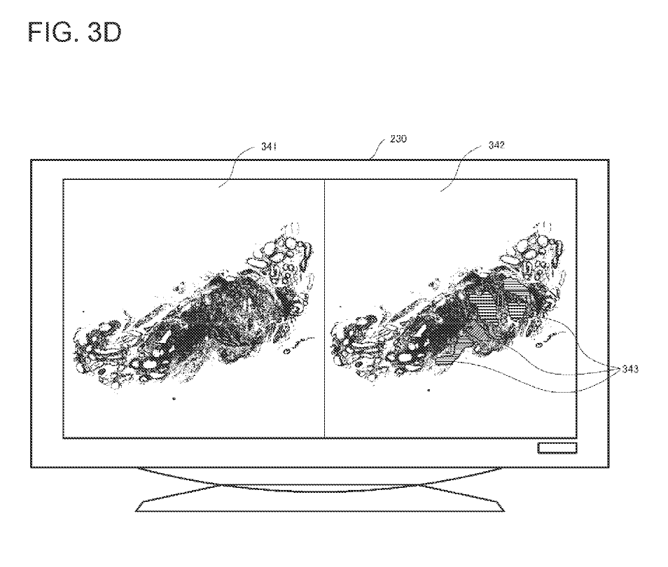

A superimposed display of the overlay image on the display screen of the communication terminal 230 in the present exemplary embodiment will be described with reference to FIGS. 3A to 3D.

FIG. 3A is a diagram showing a display of a tissue specimen image 311 on the communication terminal 230 according to the present exemplary embodiment. In FIG. 3A, the number of tissue specimen images 311 displayed on the communication terminal 230 of the pathologist is one, but is not limited thereto.

FIG. 3B is a diagram showing a display of an overlay image 321 according to the present exemplary embodiment on the communication terminal 230. The overlay image 321 is formed by generating respective areas (within predetermined ranges), each of which has the same level of the feature value obtained by analyzing the feature values of the tissue specimen image 311 of FIG. 3A, and allocating an image corresponding to the level. In addition, FIG. 3B represents the differences in levels and feature values by an oblique line, a vertical line, and a horizontal line, the densities and the thicknesses of the lines, and the like, but it is desirable to represent the differences in levels and feature values by difference in hue or difference in brightness of color, because this facilitates the determination by the pathologist. Since the drawings of the specification are not colored, the following difference in a pattern of the lines is intended to include the difference in color. Further, the combination of the pattern and the color representing the pattern can further facilitate the differentiation by pathologist. In addition, in a case of the pattern, a degree of attention of the pathologist may be differentiated by different patterns, and the same pattern is not particularly needed.

FIG. 3C is a diagram showing a display of a tissue specimen image 331 on which the overlay image according to the present exemplary embodiment is superimposed, on the communication terminal 230. In FIG. 3C, a portion of the superimposed areas in the overlay image 321 is denoted as 332. Since the pathologist can understand at a glance from the display screen shown in FIG. 3C, the feature value and the level thereof which are information of an area determined to be diagnosed in more detail and of an area to be expanded and diagnosed, the display screen shown in FIG. 3C is very useful for the pathological diagnosis.

FIG. 3D is a diagram showing displays of the tissue specimen image, and a tissue specimen image on which the overlay image according to the present exemplary embodiment of the present invention is superimposed, as another example, on the communication terminal 230. On the display screen of the communication terminal 230 shown in FIG. 3D, the tissue specimen image 341 on which the overlay image is not superimposed and the tissue specimen image 342 on which the overlay image is superimposed are displayed side by side so as to be compared. In FIG. 3D, a portion of the superimposed areas in the overlay image 321 is denoted as 343.

### (Feature Value DB)

The following is an example of the feature value DB 215 that is prepared in advance to analyze the feature value with reference to FIGS. 4A to 4E.

FIG. 4A is a diagram showing a configuration example 215-1 of the feature value DB 215 according to the present exemplary embodiment. FIG. 4A is the configuration example 215-1 of the feature value DB when the feature value is regarded at a nuclear grade which is an evaluation based on the size and the shape of the cell nucleus.

The configuration example 215-1 of the feature value DB stores in association with each body part, conditions such as a size 411 of the nucleus, a uniformity 412 of the nucleus, a distribution 413 of a chromatin, a distribution 414 of a nucleolus, and a shape 415 of the nucleus, and a score 410 for a nuclear grade (magnitude of feature value) with which the above conditions are associated.

FIG. 4B is a diagram showing a configuration example 215-2 of the feature value DB 215 according to the present exemplary embodiment. FIG. 4B is the configuration example 215-2 of the feature value DB in the case where the feature value is regarded as a degree of differentiation representing a degree of differentiation of a cancer area.

The configuration example 215-2 of feature value DB stores in association with each body part, conditions such as an array 421 of cells, a shape 422 of a gland tube, and a size disparity 423 of a nucleus, and a score 420 for a degree of differentiation (magnitude of feature value) with which the above conditions are associated. Generally, the degree of differentiation is classified into a high differentiated state, a medium differentiated state, and a low differentiated state which are obtained by dividing the level. In this case, the level is already divided, and thus images may be allocated to the divided states as they are.

FIG. 4C is a diagram showing a configuration example 215-3 of the feature value DB 215 according to the present exemplary embodiment. FIG. 4C is the configuration example 215-3 of the feature value DB in the case where the feature value is regarded as a gland tube grade, which is a structural grade, which is an evaluation of a gland tube or the like formed from a plurality of cells.

The configuration example 215-3 of the feature value DB stores in association with each body part, conditions such as a shape 431 of a gland tube including a tubular shape or a linear shape, the number 432 of cell nuclei in the gland tube, a distribution 433 of cell nuclei in a bottom portion area, and a score 430 for a structural (gland tube) grade (magnitude of feature value) with which the above conditions are associated. The details of such gland tube grade are described in Japanese Unexamined Patent Publication No. 2010-281636.

FIG. 4D is a diagram showing a configuration example 215-4 of the feature value DB 215 according to the present exemplary embodiment. FIG. 4D is the configuration example 215-4 of the feature value DB in the case where the feature value is regarded as a degree of mucus, which is an evaluation of the mucus area in the lesion.

The configuration example 215-4 of the feature value DB stores in association with each body part, conditions such as a percentage 441 of mucus present in the lesion, a percentage or distribution 442 of tissues floating in the mucus other than the mucus and a signet ring cell-like grade 443, and a score 440 for a degree of mucus (magnitude of feature value) with which the above conditions are associated. In addition, see, for example, Patent Document 1 for an extraction method of a mucus area.

FIG. 4E is a diagram showing a configuration example 215-5 of the feature value DB 215 according to this exemplary embodiment of the present invention. FIG. 4E is a configuration example 215-5 of a feature value DB 215 in the case where the feature value is regarded as a histological grade that is an evaluation of a histopathological grade of total cancer cells including a nuclear grade of FIG. 4A, and the like.

The configuration example 215-5 of the feature value DB stores in association with each body part, conditions such as a nuclear grade 451 and the number of occurrences of nuclear fission 452, and a score 450 for a nuclear grade (magnitude of feature value) with which the above conditions are associated. Further, the configuration example 215-5 stores a condition of a structural grade 461 and a score 460 for a histological grade (magnitude of feature value) with which the above conditions are associated, in addition to the nuclear grade 451 and the number of occurrences of nuclear fission 452. In addition, as the structural grade 461, for example, a degree of a gland tube formation is included.

### (Level Classification DB)

FIG. 5 is a diagram showing a configuration of a level classification DB 217 according to the present exemplary embodiment. Further, in practice, the score of the feature value may be divided into different levels depending on the body part or the like, and FIG. 5 shows the example.

The level classification DB 217 stores a feature value 501 including each feature value or a combination of a plurality of feature values, and a level value associated with the score range 502. FIG. 5 shows an example of a level of ten stages, but the level is not limited thereto. For example, if there are high, medium and low differentiations, the level has three stages.

### (Allocation Image DB)

FIG. 6 is a diagram showing a configuration of an allocation image DB 219 according to the present exemplary embodiment. Further, the present exemplary embodiment shows an example in which the pathologist identifies the color and the pattern from the displayed image, but other identifiable examples can be also applied. Further, FIG. 6 also gives an example of having the level of ten stages, but the level is not limited thereto.

The allocation image DB 219 stores color information in association with the level 601. In FIG. 6, as an example of color, a first hue group 602, a second hue group 603, brightness of Red (R) 604, brightness of Green (G) 605, and brightness of Blue (B) 606 are stored. The hue groups are not limited to the combinations of the present example, and the brightnesses may be those of other mixtures of color.

Further, the pattern information is stored in association with the level 601. In FIG. 6, as an example of the pattern, hatched patterns are stored as the first pattern group 607 and horizontal line patterns are stored as the second pattern group 608. Further, the patterns are not limited to the examples of FIG. 6. However, since it is difficult to determine the difference of levels of complex patterns, a simple pattern is desirable.

### (Area Information)

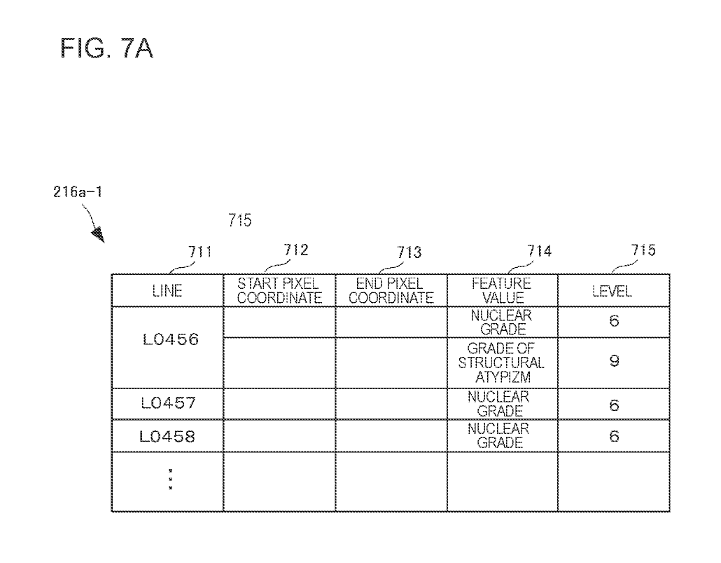

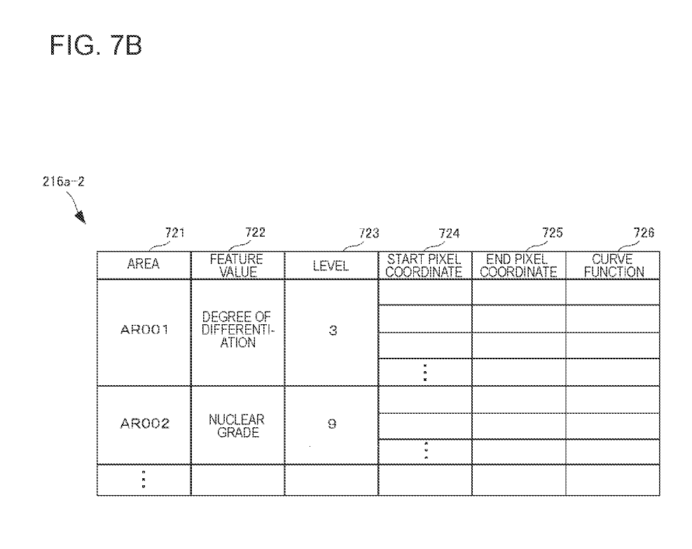

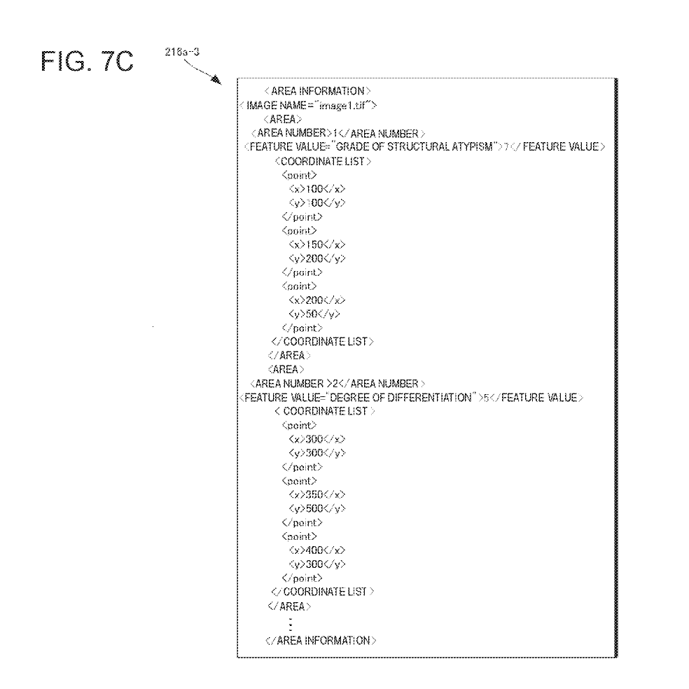

An example of the area information 216a which is output to the overlay image generation unit 218 by the area generation unit 216 will be described with reference to FIGS. 7A to 7C. In addition, the area information may be information which is developed into the bit map, but this increases the amount of information and affects the processing speed of the device, and therefore it is desirable that the area information be data in display line units or area units as in the following example.

FIG. 7A is a diagram showing an example 216a-1 of the area information 216a according to the present exemplary embodiment. In the present example, data indicating an area in display line unit is used.

In an example of area information 216a-1, in association with a line 711, a start pixel coordinate 712 and an end pixel coordinate 713 which are contained in an area on the line are stored, and the feature value 714 and the level 715 of the area are stored. In addition, as the line 711, all lines crossing the area generated by the area generation unit 216 are stored.

FIG. 7B is a diagram showing another example 216a-2 of the area information 126a according to the present exemplary embodiment. In the present example, data shows an area with vectors in area units. That is, in the present example, the outline of the area is represented with vectors.

In another example of area information 216a-2, in association with an area 721, a feature value 722 and a level 723 are stored, singularities for forming the area are stored as a start pixel coordinate 724 and an end pixel coordinate 725, and a curve function 726 connecting the singularities is stored. In addition, the curve function 726 may be stored as, for example, a spline curve or the like and the parameters thereof. In the present example, only the area generated by the area generation unit 216 is stored.

FIG. 7C is a diagram showing further another example 216a-3 of an area information 126a according to the present exemplary embodiment. The example of FIG. 7C is the example representing the area in the text data of XML format. In addition, since text data written in XML format is well known, a detailed description thereof is not repeated.

### (Overlay Image Information)

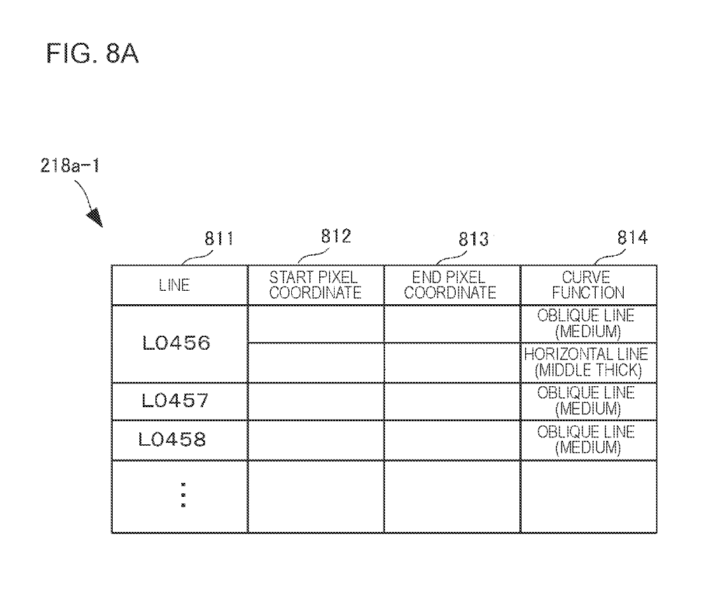

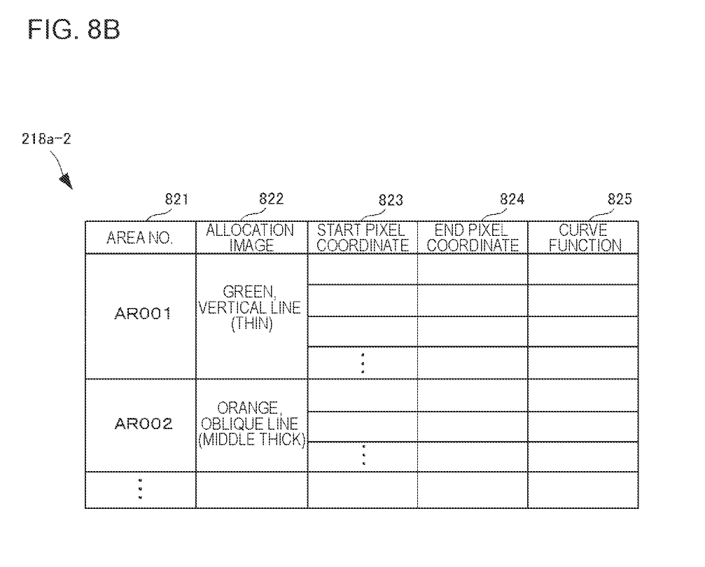

FIGS. 8A and 8B are examples of overlay image information which are generated based on the area information of FIGS. 7A and 7B. In addition, the overlay image information may be information which is developed into the bit map, but this increases the amount of information and affects the communication traffic, and therefore it is desirable that the overlay image information be data in display line units or area units as in the following example. In addition, the overlay image may be overlay image information which is written in the format generated based on the text data written in XML format shown in FIG. 7C (not shown).

FIG. 8A is a diagram showing an example 218a-1 of the overlay image information 218a according to the present exemplary embodiment. The present example is overlay image information in display line units corresponding to the area information of FIG. 7A.

In an example 218a-1 of the overlay image information, in association with a line 811, a start pixel coordinate 812 and an end pixel coordinate 813 included in an area of the line are stored, and the allocation image 814 allocated to the overlay image generation unit 218 is stored in the area. In addition, as the line 811, all lines crossing the area generated by the area generation unit 216 are stored and transmitted to the communication terminal 230.

FIG. 8B is a diagram showing another example of overlay image information according to the present exemplary embodiment. The present example is overlay image information in area units corresponding to area information of FIG. 7B.

In an example of overlay image information 218a-2, in association with an area 821, the allocation image 822 allocated to the area by the overlay image generation unit 218 is stored, singularities forming the area are stored as a start pixel coordinate 823 and an end pixel coordinate 824, and a curve function 825 connecting the singularities is stored. In addition, the curve function 825 may be stored, for example, as a spline curve and the parameters thereof. In the present example, only the areas generated by the area generation unit 216 are stored and transmitted to the communication terminal 230.

### <<Hardware Configuration of Information Processing Apparatus>>

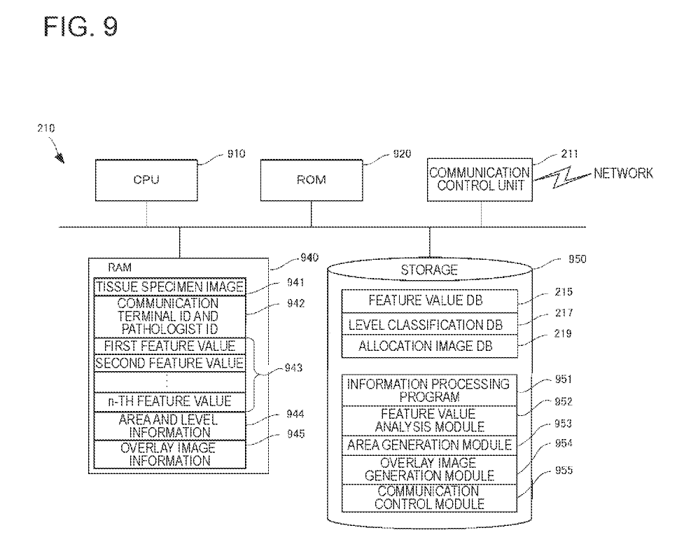

FIG. 9 is a block diagram showing a hardware configuration of an information processing apparatus 210 according to the present exemplary embodiment.

In FIG. 9, a CPU 910 is an operation control processor, and implements a program so as to realize each functional configuration unit of FIG. 2. A ROM 920 stores fixed data and programs such as initial data and programs. A communication control unit 211 communicates with the communication terminal 230 of the pathologists. In addition, communication may be either wired or wireless.

A RAM 940 is a random access memory used by the CPU 910 as a work area for temporary storage. An area for storing data required for realizing the present exemplary embodiment is ensured in the RAM 940. 941 is an area for storing the tissue specimen image that has been received through the network 250 from the communication terminal 230 of the pathologist. 942 is an area for storing information for specifying the tissue specimen image 941 such as a communication terminal ID of the communication terminal 230 that has transmitted the tissue specimen image 941 and a pathologist ID. The information 942 for specifying the tissue specimen image 941 includes for example, a patient ID, the body part from which a tissue specimen is taken, gender, age, medical history, or the like. 943 is an area for storing the feature value which is calculated by the feature value analysis. 944 is an area for storing levels classified on the basis of the feature value 943 which is calculated, and the information of an area having the level (see FIGS. 7A to 7C). 945 is an area for storing the overlay image information transmitted to the communication terminal 230 of the communication terminal ID (see FIGS. 8A and 8B).

A storage 950 stores databases and various parameters, or the following data or programs required for realizing the present exemplary embodiment. 215 is a feature value DB (see FIGS. 4A to 4E). 217 is a level classification DB (see FIG. 5). 219 is an allocation image DB (see FIG. 6). The following programs are stored in the storage 950. 951 is an information processing program which is a pathological diagnosis support program for performing all processes. 952 is a feature value analysis module which analyzes the feature value of the tissue specimen image in the information processing program 951. 953 is an area generation module which generates an area of the same feature value and the same level in the information processing program 951. 954 is an overlay image generation module which allocates an identifiable image to an area of the same feature value and the same level and generates an overlay image, in the information processing program 951. 955 is a communication control module which controls communication with the communication terminal 230 by the communication control unit 211, in the information processing program 951.

In addition, in FIG. 9, only the data and programs required in the present exemplary embodiment are shown, but data and programs of general-purpose such as OS are not shown.

### <<Processing Procedure of Information Processing Apparatus>>

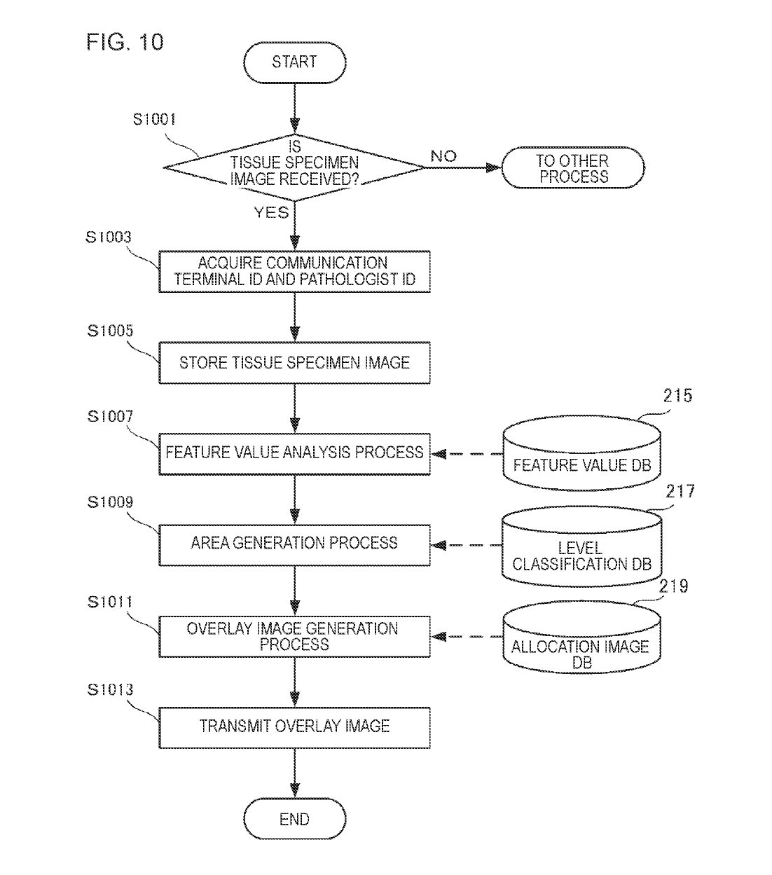

FIG. 10 is a flowchart showing a processing procedure of the information processing apparatus 210 according to the present exemplary embodiment. In the flowchart, the CPU 910 of FIG. 9 realizes functional configuration units of the information processing apparatus 210 of FIG. 2 while using the RAM 940.

First, in step S1001, the information processing apparatus 210 determines whether or not the received data is a tissue specimen image received from any one of the communication terminals 230. If the received data is not the tissue specimen image, the process proceeds to another process.

If the received data is the tissue specimen image, the process proceeds to step S1003, and the information processing apparatus 210 acquires the communication terminal ID (for example, IP address, or the like) of the communication terminal 230 which has transmitted the tissue specimen image, and the information (pathologist IDs, patient IDs, body parts, and the like) for specifying the tissue specimen image. In step S1005, the information processing apparatus 210 stores the received tissue specimen image.

In step S1007, the information processing apparatus 210 performs the analysis process on the feature value while referring to the feature value DB 215. Next, in step S1009, the information processing apparatus 210 performs the area generation process while referring to the level classification DB 217. Next, in step S1011, the information processing apparatus 210 performs the overlay image generation process while referring to the allocation image DB 219. Then, in step S1013, the information processing apparatus 210 transmits the generated overlay image back to the communication terminal 230 which has transmitted the tissue specimen image.

In general, when the tissue specimen image is acquired for supporting the pathological diagnosis, a tissue specimen image of low resolution is first acquired for performing a rough diagnosis, and then a tissue specimen image of high resolution is acquired if a detailed diagnosis is required. The procedure also may be applied to the procedure of the present exemplary embodiment. Alternatively, if support for giving a tip for the detailed diagnosis of the pathologist is wanted, an overlay image may be generated only from the tissue specimen image of low resolution. In contrast, if support for showing the direction of diagnosis or to evaluate the result of diagnosis of the pathologist is wanted, it is preferable that an overlay image be generated by performing a preliminary diagnosis using the tissue specimen image of high resolution.

### Third Exemplary Embodiment

Next, an information processing system according to the third exemplary embodiment of the present invention will be described. The information processing system according to the present exemplary embodiment is different from that of the second exemplary embodiment in that there are three feature values to be analyzed, and Red (R), Green (G) and Blue (B), three primary colors of light, are allocated to the three feature values. As a result, combinations of the levels of three features are displayed in different colors. In addition, although Red is allocated to the feature value of a cell, Green is allocated to the feature value of a gland tube and Blue is allocated to the feature value of mucus in the present exemplary embodiment, the three feature values and the allocation of colors are not limited to those of the present example.

According to the present exemplary embodiment, the levels of three feature values can be determined at the same time from a tendency of color (reddish, bluish, whitish, and the like). Therefore, proper selection of three feature values and allocation of colors enhance the comprehensive determination from the hue by a plurality of features.

In addition, only the characteristic configuration of the present exemplary embodiment is described, other configurations and operations are the same as in the second exemplary embodiment, and thus the detailed description is not repeated.

### <<Configuration of an Information Processing System>>

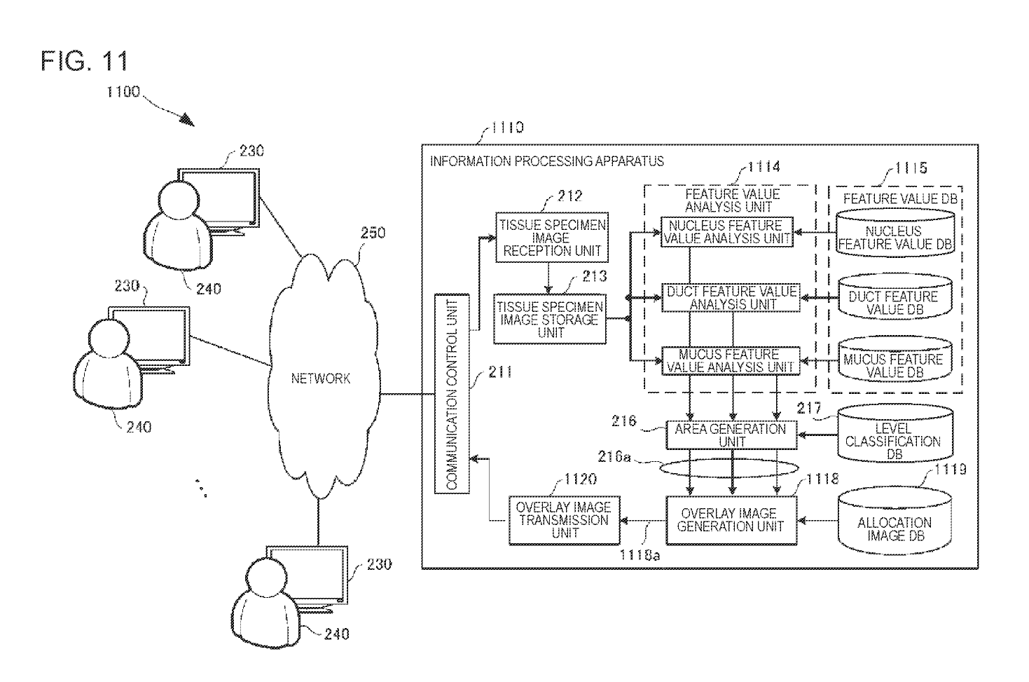

FIG. 11 is a block diagram showing a configuration of an information processing system 1100 according to the present exemplary embodiment. In addition, the same constituent elements and the functional configuration units of the information processing apparatus as in the second exemplary embodiment are denoted by the same reference numerals, and thus the description thereof is not repeated.

The information processing apparatus 1110 of FIG. 11 analyzes three feature values, allocates three primary colors of light to each feature value, and associates the level of the feature value with brightness so as to generate three overlay images. Thus, a combination of two feature values, the level display of any one feature value, and the like are possible by a simple operation (a removal operation and an addition operation of three overlay images).

In a feature value analysis unit 1114 and a feature value DB 1115 of the information processing apparatus 1110, the feature value is limited to three feature values: a nuclear feature value, a gland tube feature value and a mucus feature value, but there is no significant difference in the configuration.

The overlay image generation unit 1118 generates three overlay images with reference to the stored allocation image DB 1119, based on the allocation of three feature values and three primary colors which are selected in advance. The overlay image transmission unit 1120 transmits the three generated overlay images to the communication terminal 230 through the network 250.

### (Allocation Image DB)

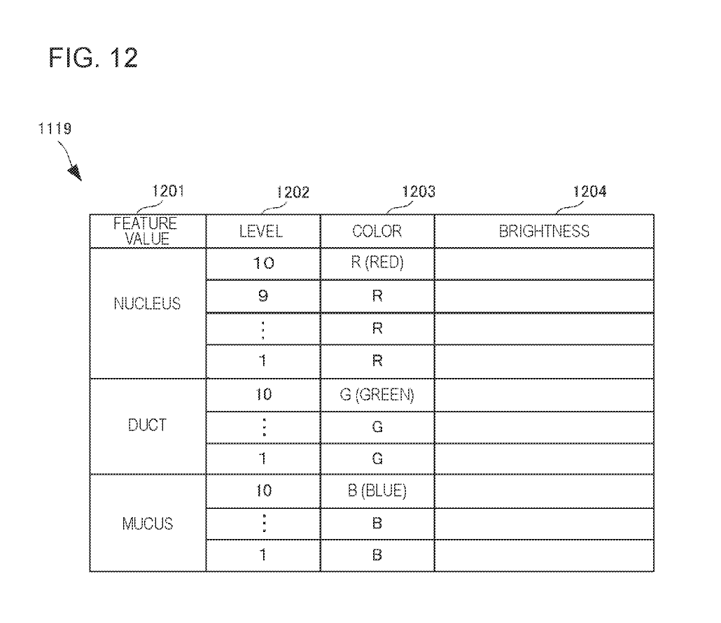

FIG. 12 is a diagram showing a configuration of an allocation image DB 1119 according to the present exemplary embodiment.

In the allocation image DB 1119, the color 1203 and the brightness 1204 of three primary colors are stored in association with a feature value 1201 and a level 1202. In the present example, as the feature value 1201, a nucleus, a gland tube and mucus are stored and respectively associated with Red (R), Green (G) and Blue (B).

### (Overlay Image Information)

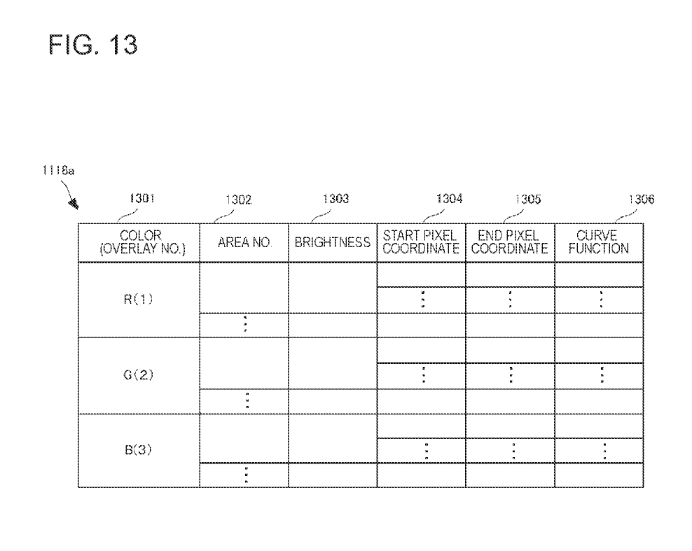

FIG. 13 is a diagram showing overlay image information 1118a according to the present exemplary embodiment. In addition, an example of an outline view of an area with vectors shown in FIG. 8B is applied to the overlay image information 1118a of FIG. 13.

In the overlay image information 1118a, a generated area 1302 is stored for each overlay number 1301 for identifying three overlay images corresponding to three primary colors. A brightness 1303, a start pixel coordinate 1304 and an end pixel coordinate 1305, which represent an outline of an area, and a curve function 1306 are stored in association with the area 1302.

### Fourth Exemplary Embodiment

Next, an information processing system according to the fourth exemplary embodiment of the present invention will be described. The information processing system according to the present exemplary embodiment is different from that of the second exemplary embodiment in that the pathologist 240 can select the feature value to be analyzed and the allocation image to be allocated to the feature value from the communication terminal 230. In addition, although the present exemplary embodiment shows a configuration in which the pathologist can select both the feature value and the allocation image, a configuration in which only one thereof can be selected is possible.

According to the present exemplary embodiment, it is possible to analyze and display the feature value which is desired by the pathologist and to allow the pathologist to determine at a glance the feature value and the level to which the pathologist has to pay attention.

In addition, only the characteristic configuration of the present exemplary embodiment is described, other configurations and operations are the same as in the second exemplary embodiment, and thus the detailed description is not repeated.

### <<Configuration of an Information Processing System>>

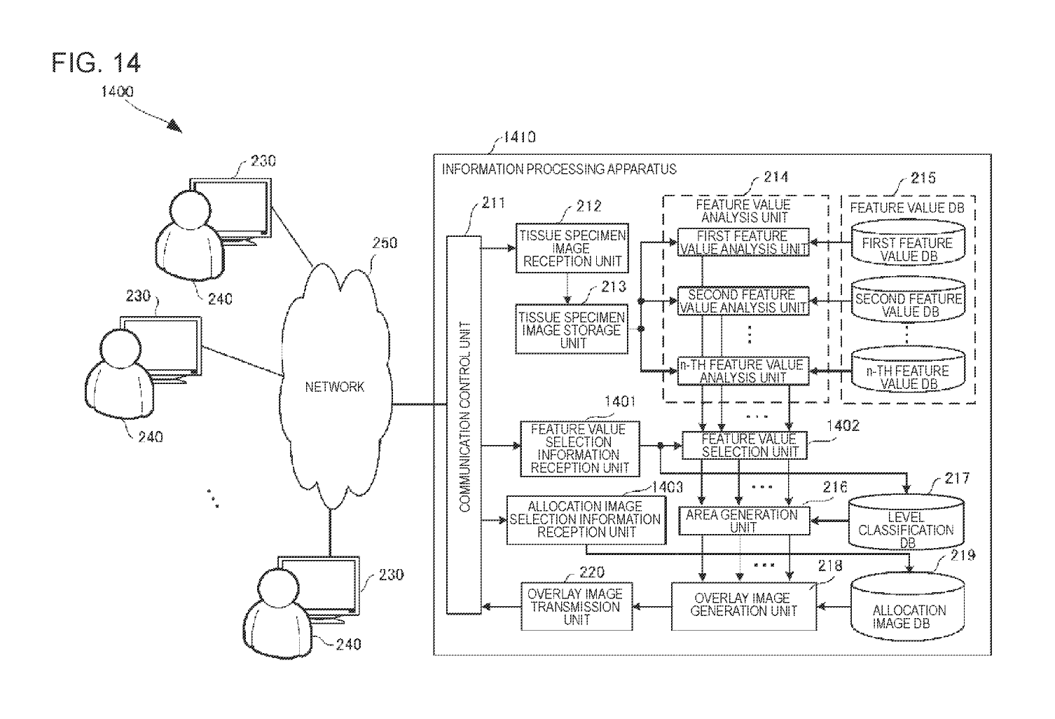

FIG. 14 is a block diagram showing a configuration of an information processing system 1400 according to the present exemplary embodiment. In addition, the same constituent elements and the functional configuration units of the information processing apparatus as in the second exemplary embodiment are denoted by the same reference numerals, and thus the description thereof is not repeated.

A feature value selection information reception unit 1401 of the information processing apparatus 1410 receives information of the feature value selection instruction by the pathologist 240 that is transmitted from the communication terminal 230 through the network 250. The feature value selection unit 1402 analyzes the feature value according to the selection by the pathologist 240, which is received by the feature value selection information reception unit 1401.

Further, an allocation image selection information reception unit 1403 receives the information of the allocation image selection instruction by the pathologist 240 which is transmitted from the communication terminal 230 through the network 250. The result of reception is reported to the allocation image DB 219, the allocation image selected by the pathologist 240 is associated with each feature value, and the overlay image is generated.

### (Screen for Selecting a Feature Value and a Level Image)

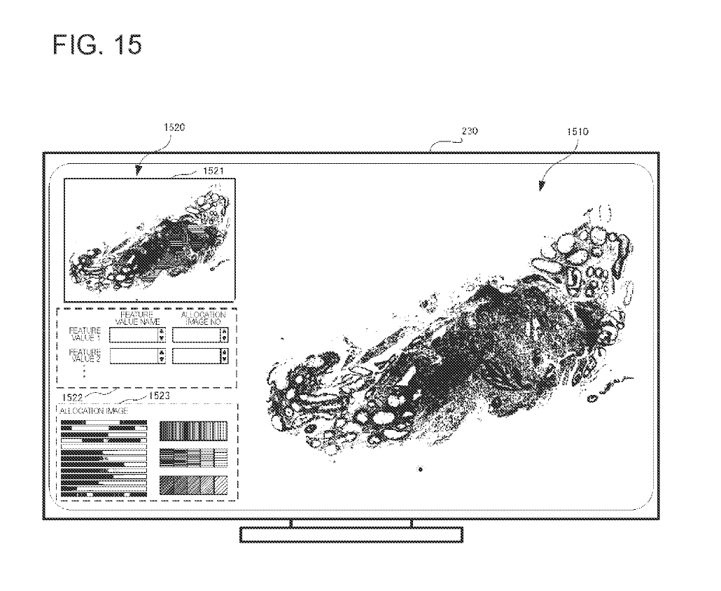

FIG. 15 is a diagram showing a screen for selecting a feature value and a level image according to the present exemplary embodiment, in the communication terminal 230. FIG. 15 is only an example and the screen for selecting a feature value and a level image is not limited thereto.

1510 in FIG. 15 is a display area of the transmitted tissue specimen image. 1520 is a display area that can interact with the information processing apparatus 1410. 1522 is a selection instruction area for inquiring about the feature value and the allocation image from the information processing apparatus 1410, in response to the transmission of the tissue specimen image. 1523 is a list of allocation images. In the list of allocation images 1523, a hue group is shown on the left and a pattern group is shown on the right. The number of levels is not limited thereto. 1521 is a display image in which the overlay image generated from the transmitted tissue specimen image in the information processing apparatus 1410 is superimposed on the tissue specimen image, according to the selection from the selection instruction area 1522.

### <<Operation Procedure of Information Processing System>>

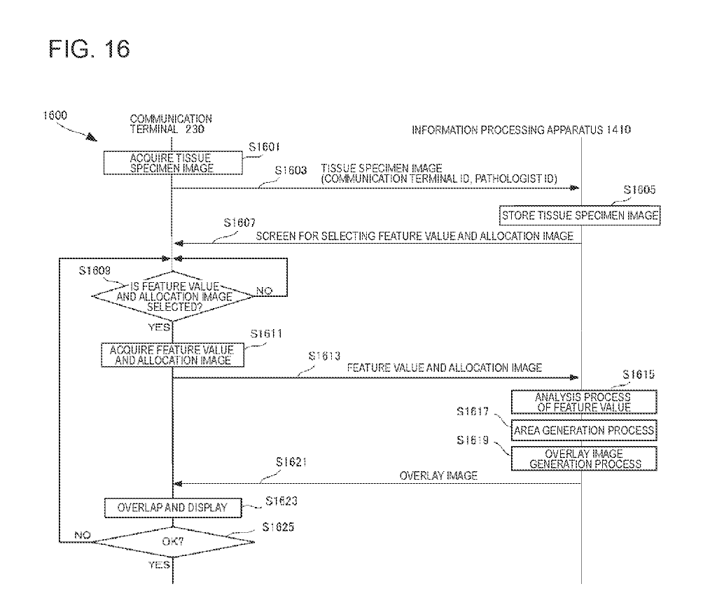

FIG. 16 is a sequence diagram showing an operation procedure 1600 of the information processing system according to the present exemplary embodiment.

First, in step S1601, the communication terminal 230 acquires a tissue specimen image. The tissue specimen image may be read and acquired from a scanner (not shown) connected to the communication terminal 230 or acquired through the storage medium or the like. In step S1603, the communication terminal 230 transmits the acquired tissue specimen image to the information processing apparatus 1410. The information processing apparatus 1410 stores the tissue specimen image received in step S1605. Subsequently, in step S1607, the information processing apparatus 1410 transmits a screen for inquiring about the feature value selection and the allocation of the allocation image to the communication terminal.

The communication terminal 230 is on standby for the feature value selection and the allocation image selection by the pathologist 240 in step S1609, and if there is selection, the process proceeds to step S1611. The communication terminal 230 acquires the information on the feature value and the allocation image which are selected in step S1611, and transmits the acquired information back to the information processing apparatus 1410 in step S1613.

The information processing apparatus 1410 performs an analysis process of the feature value which is selected by the pathologist 240 in step S1615. Subsequently, the information processing apparatus 1410 performs an area generation process of a level corresponding to the feature value in step S1617. Next, the information processing apparatus 1410 performs a generation process of an overlay image, which is allocated with the allocation image selected by the pathologist 240, to each area in step S1619. Then, the information processing apparatus 1410 transmits the overlay image generated in step S1621 according to the feature value and the allocation image which are selected by the pathologist 240, to the communication terminal 230.

The communication terminal 230 superimposes the tissue specimen image that has been transmitted on the received overlay image and displays the superimposed image, in step S1623. The pathologist 240 determines an area to be further diagnosed in detail and an area to be expanded and displayed, with reference to the displayed superimposed image. In addition, in step S1625, the pathologist 240 determines whether or not the displayed superimposed image is a desired result, and in a case of selecting again different feature values or allocation images, the process is returned to step S1609 and is repeated, by operating the communication terminal 230.

In addition, the tissue specimen image may be transmitted simultaneously with the feature value selection information and the allocation image selection information. Further, an inquiry for feature value selection information and an inquiry for allocation image selection may be performed in different steps.

### <<Hardware Configuration of an Information Processing Apparatus>>

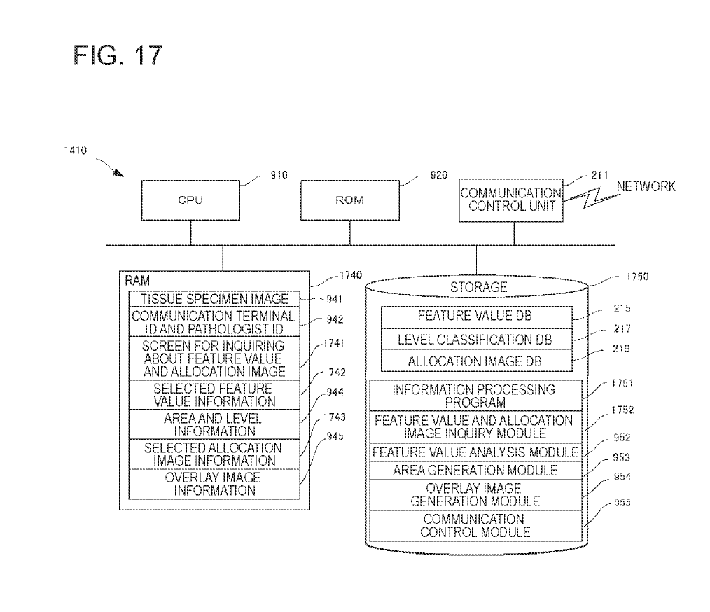

FIG. 17 is a block diagram showing a hardware configuration of the information processing apparatus 1410 according to the present exemplary embodiment. In addition, in FIG. 17, constituent elements that perform the same function as the configuration of FIG. 9 of the second exemplary embodiment are denoted by the same reference numerals, and thus the description thereof is not repeated.

FIG. 17 is different from FIG. 9 in the configurations of a RAM 1740 and storage 1750.

First, the RAM 1740 is different from the RAM in FIG. 9 in a screen 1741 (see FIG. 15) for inquiring about the feature value and allocation image of the communication terminal 230. Then, the difference is in selected feature value information 1742 and selected allocation image information 1743 which are selected by the pathologist 240 and transmitted from communication terminal 230.

Further, the storage 1750 is different from the storage in FIG. 9 in the change of an information processing program 1751 which is a pathological diagnosis support program. The change mainly results from a feature value and allocation image inquiry module 1752 which inquires about the feature value and the allocation image of the pathologist 240.

### <<Processing Procedure of Information Processing Apparatus>>

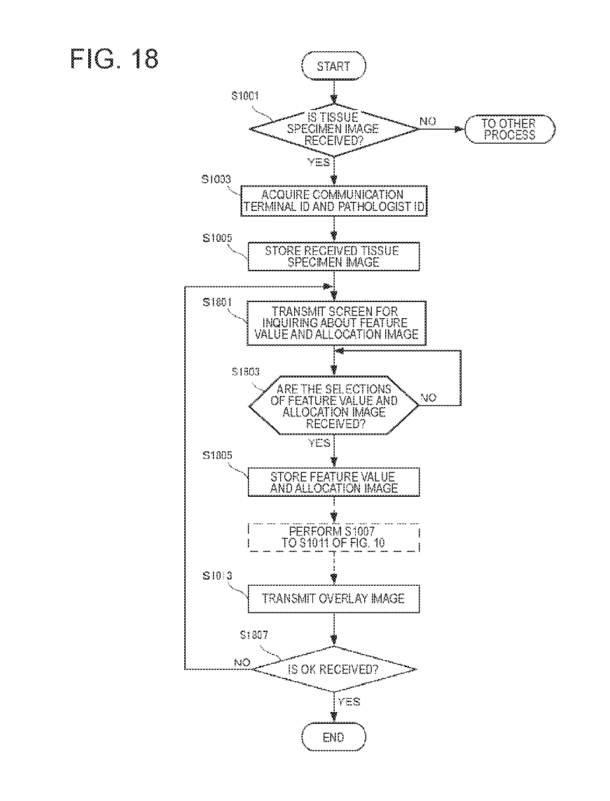

FIG. 18 is a flowchart showing a processing procedure of an information processing apparatus 1410 according to the present exemplary embodiment. In the flowchart, the CPU 910 of FIG. 17 realizes functional configuration units of the information processing apparatus 1410 of FIG. 14 while using the RAM 1740. In addition, in FIG. 18, the steps of performing the same process as in FIG. 10 of the second exemplary embodiment are denoted by the same step numbers, and thus the description thereof is not repeated.

The information processing apparatus 1410 transmits a screen for inquiring about a feature value and an allocation image to the communication terminal 230, in step S1801. Then, in step S1803, the information processing apparatus 1410 is on standby for receiving the selection information for selecting the feature value and the allocation image from the communication terminal 230, and if there is reception, the process proceeds to step S1805. In step S1805, the information processing apparatus 1410 stores the selection information for selecting the received feature value and allocation image. In subsequent steps S1007 to S1011, the information processing apparatus 1410 performs respective processes including a feature value analysis, an area generation, and an overlay image generation using the feature value and the allocation image which are selected by the pathologist 240. In step S1013, the information processing apparatus 1410 transmits the generated overlay image to the communication terminal 230.

In step S1807, the information processing apparatus 1410 is on standby for input from the pathologist 240 as to whether or not the input is “OK”, a result of determination of whether the desired result is achieved from the selection of the feature value and the selection of the allocation image. If the input is not OK, the process returns to step S1801, and the information processing apparatus 1410 is again on standby for the selection information of the feature value and the allocation image from the communication terminal 230, and repeats the process described above.

### Fifth Exemplary Embodiment

Next, an information processing system according to the fifth exemplary embodiment of the present invention will be described. An information processing system according to the present exemplary embodiment is different from the fourth exemplary embodiment in that the selection of the feature value and the allocation image are not performed by the pathologist 240 but performed automatically by the information processing apparatus based on the specific information of a tissue specimen image.

According to the present exemplary embodiment, without the selection by the pathologist, since the desired feature value and the desired allocation image are suitably selected from the tissue specimen image, the feature value and the level to which the pathologist has to pay attention can be objectively determined at a glance.

In addition, only the characteristic configuration of the present exemplary embodiment is described, and other configurations and operations are the same as in the fourth exemplary embodiment. Therefore, the detailed description is not repeated.

### <<Configuration of an Information Processing System>>

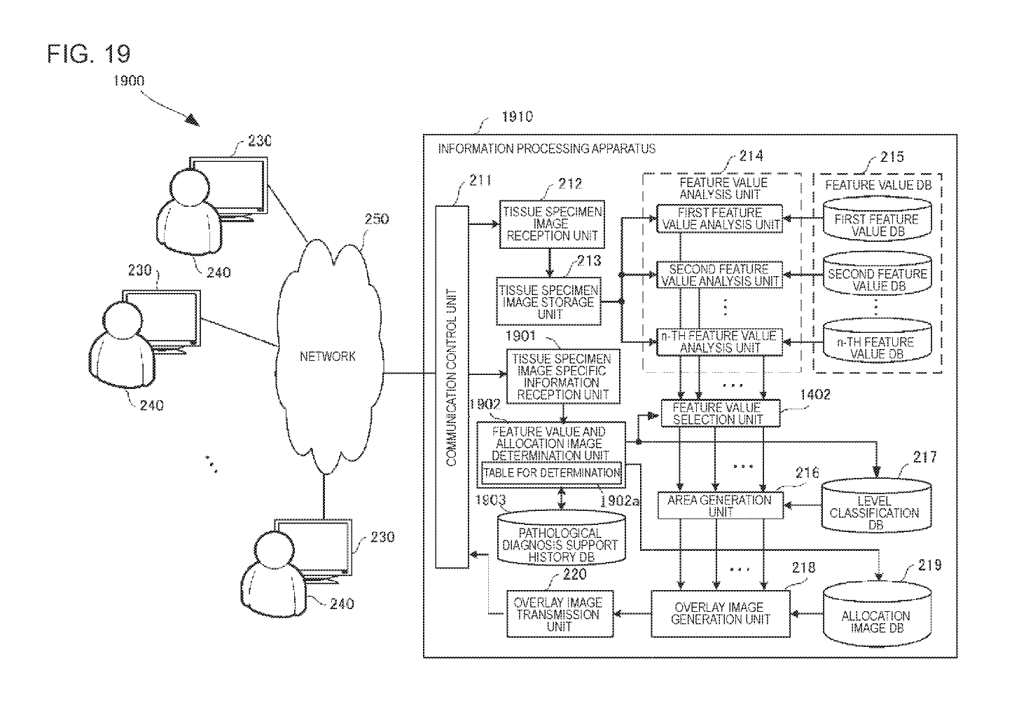

FIG. 19 is a block diagram showing a configuration of the information processing system 1900 according to the present exemplary embodiment. In addition, the same constituent elements and the functional configuration units as in the fourth exemplary embodiment are denoted by the same reference numerals, and thus the description thereof is not repeated.

The tissue specimen image specific information reception unit 1901 of the information processing apparatus 1910 receives specific information for specifying the tissue specimen image which is transmitted from the communication terminal 230 through the network 250. The specific information includes a pathologist ID, a patient ID, body parts, gender, age, medical history, and the like. In addition, a configuration is possible in which the information processing apparatus 1910 can acquire other information from the pathological diagnosis support history DB 1903, based on the pathologist ID and the patient ID.

With reference to the pathological diagnosis support history DB 1903, a feature value and allocation image determination unit 1902 automatically determines the feature value and the allocation image from the received specific information, using a table for determination 1902a. The feature value and allocation image determination unit 1902 selects the feature value in the feature value selection unit 1402 according to the determined feature value and the allocation image, and selects the allocation image allocated from the allocation image DB 219.

### (Screen for Specifying Tissue Specimen Image)

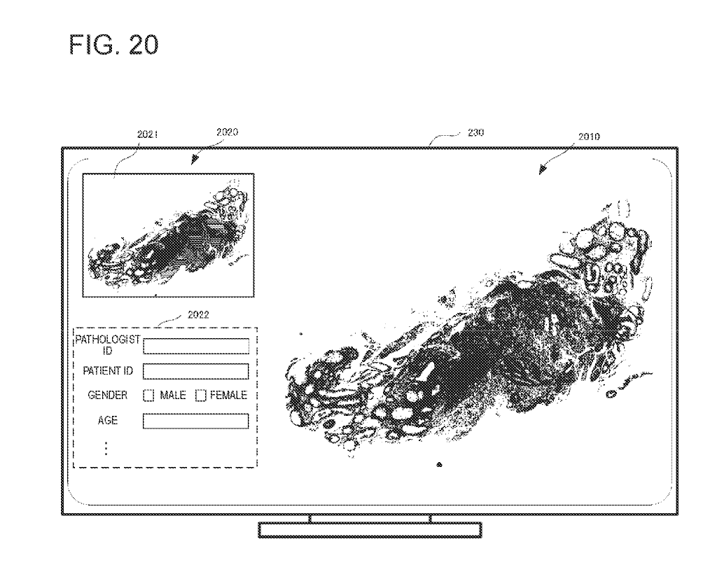

FIG. 20 is a diagram showing a screen for specifying a tissue specimen image according to the present exemplary embodiment, in the communication terminal 230. FIG. 20 is an example, and a screen for specifying a tissue specimen image is not limited thereto.

2010 in FIG. 20 is a display area of the transmitted tissue specimen image. 2020 is a display area that can interactively exchange information with the information processing apparatus 1910. 2022 is an input area of inquiring about the specific information of the tissue specimen image which is transmitted from the information processing apparatus 1910 in response to the transmission of the tissue specimen image. 2021 is a display image in which the overlay image generated from the transmitted tissue specimen image in the information processing apparatus 1910 according to the selection from the input area 2022 is superimposed on the tissue specimen image.

### (Table for Determination)

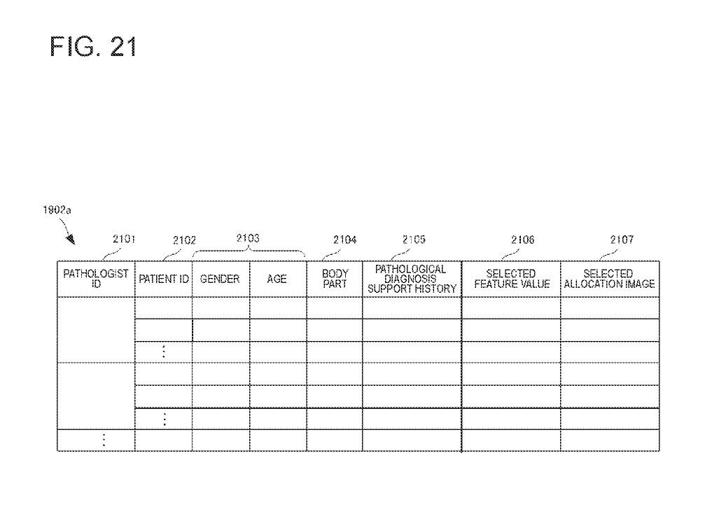

FIG. 21 is a diagram showing a configuration of a table for determination 1902a according to the present exemplary embodiment.

The table for determination 1902a stores a selected feature value 2106 and a selected allocation image 2107 in association with a pathologist ID 2101, a patient ID 2102, a patient attribute 2103, a body part 2104 to be taken, and a pathological diagnosis support history 2105. Based on the table for determination 1902a, the feature value and allocation image determination unit 1902 determines the selected feature value and the selected allocation image for the received tissue specimen image.

### <<Processing Procedure of Information Processing Apparatus>>

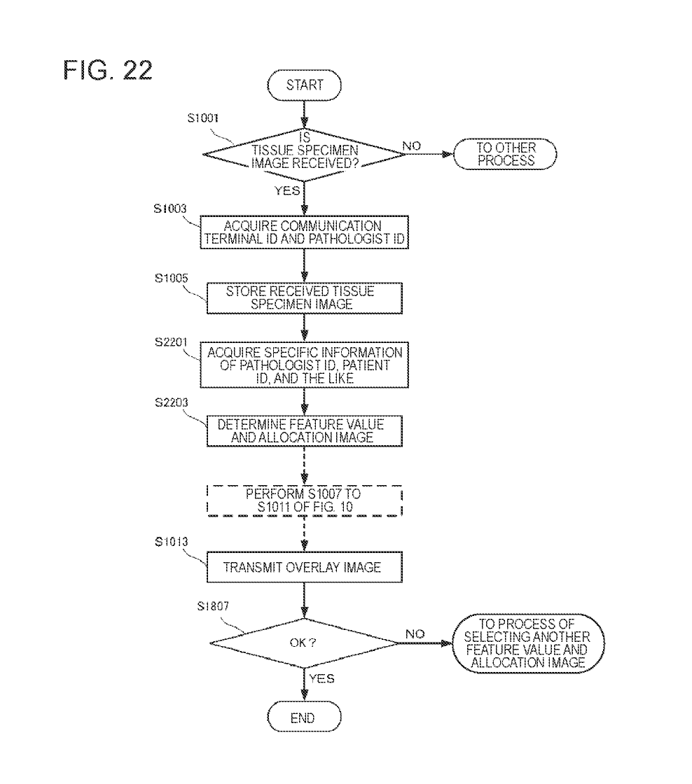

FIG. 22 is a flowchart showing a processing procedure of an information processing apparatus 1910 according to the present exemplary embodiment. In the flowchart, the CPU 910 of FIG. 17 realizes functional configuration units of the information processing apparatus 1910 of FIG. 19 while using the RAM 1740. In addition, in FIG. 22, the same steps as in FIG. 18 of the fourth exemplary embodiment are denoted by the same step numbers, and thus the description thereof is not repeated.

In step S2201, the information processing apparatus 1910 acquires specific information including the pathologist ID, the patient ID, and the like. Subsequently, in step S2203, the information processing apparatus 1910 determines a feature value and an allocation image from the acquired specific information. The following procedures are the same as in FIG. 18. In addition, in step S1807, in a case of “not OK”, the information processing apparatus 1910 stops the automatic selection, and proceeds to a process of performing reselection of other feature values and allocation images.

### Sixth Exemplary Embodiment

Next, an information processing system according to the sixth exemplary embodiment of the present invention will be described. The information processing system according to the present exemplary embodiment is different from the second exemplary embodiment in that it superimposes an overlay image on the tissue specimen image to display on the communication terminal 230, and then expands and displays the specified area with the magnification percentage according to the feature value, in response to an area expansion instruction from the pathologist 240. In addition, although the present exemplary embodiment shows an example of displaying an expanded image on a separate area of the screen of the communication terminal operated by the pathologist, the expanded image may be displayed on a separate screen or displayed on the instructed position of the tissue specimen image, as a magnifying glass.

According to the present exemplary embodiment, when the pathologist determines the feature value and the level of the tissue specimen image to which the pathologist has to pay attention and then instructs expansion and display of the desired area, it is possible to expand and display the area with the magnification according to the feature value of the instructed area. Thus, it may be possible to eliminate the need for magnification adjustment by the pathologist and to reduce labor of work.

In addition, only the characteristic configuration of the present exemplary embodiment is described, other configurations and operations are the same as in the second exemplary embodiment, and thus the detailed description is not repeated.

### <<Configuration of an Information Processing System>>

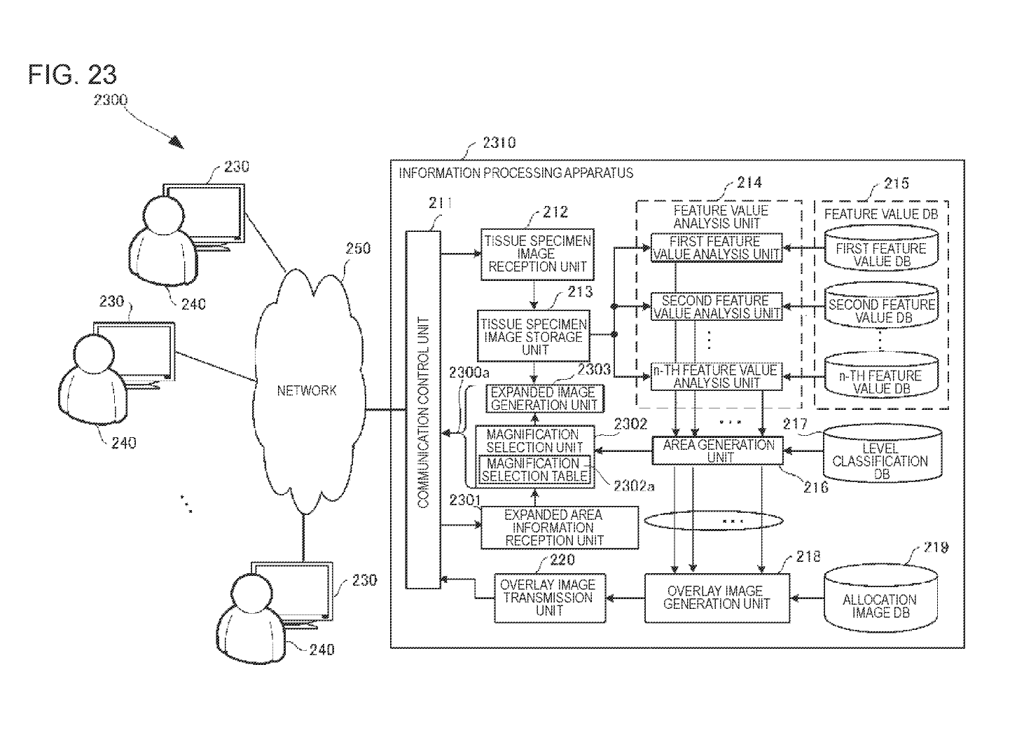

FIG. 23 is a block diagram showing a configuration of an information processing system 2300 according to the present exemplary embodiment. In addition, the same constituent elements and the functional configuration units of the information processing apparatus as in the second exemplary embodiment are denoted by the same reference numerals, and thus the description thereof is not repeated.

An expanded area information reception unit 2301 of an information processing apparatus 2310 receives an area instruction on the screen of the communication terminal 230 on which the overlay image transmitted by the overlay image transmission unit 220 is superimposed and displayed. That is, if the pathologist 240 instructs an area of the overlay image which is displayed on the communication terminal 230, the communication terminal 230 transmits the area information together with the expansion instruction to the information processing apparatus.

A magnification selection unit 2302 selects a magnification ratio using a magnification selection table 2302a, according to the feature value which is coincident with the area information received from the communication terminal 230 and corresponds to the area information from the area generation unit 216. An expanded image generation unit 2303 expands the corresponding area of the tissue specimen image according to the magnification selected by the magnification selection unit 2302. Then, the expanded image generation unit 2303 transmits expanding transmission data 2300a containing the magnification information and the expanded image of the corresponding area back to the communication terminal 230. In addition, if an application capable of expanding an area with the received magnification is operable in the communication terminal 230, the expanded image generation unit 2303 is not an essential constituent element. In the first place, since the communication terminal 230 has a tissue specimen image of the highest resolution, it is desirable to have configurations for expanding an area with the magnification corresponding to the feature value received in the communication terminal 230, in view of communication traffic.

### (Screen for Expanding an Area of a Tissue Specimen Image)

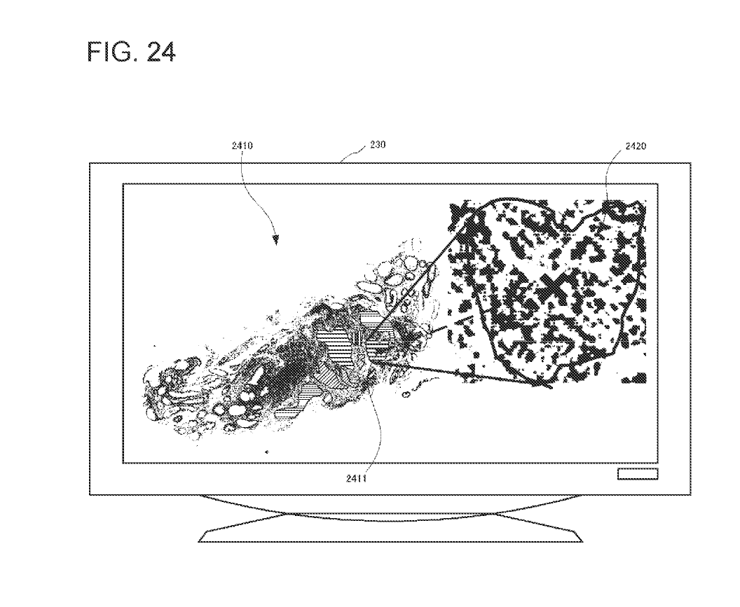

FIG. 24 is a drawing showing a screen for expanding an area of a tissue specimen image according to the present exemplary embodiment, in the communication terminal 230. FIG. 24 is an example, but the screen for expanding an area of a tissue specimen image is not limited thereto.

In FIG. 24, 2410 is an image display area in which the overlay image received from the information processing apparatus 2310 is superimposed on the tissue specimen image that has been transmitted.

It is assumed that the pathologist 240 selects the area 2411 as an area to be expanded and diagnosed in detail from the overlay image of the superimposed image. In FIG. 24, 2420 is an expanded image obtained by expanding the area 2411 with a magnification corresponding to the feature value. The magnification of FIG. 24 is not exact and does not reflect the actual magnification.

### (Magnification Selection Table)

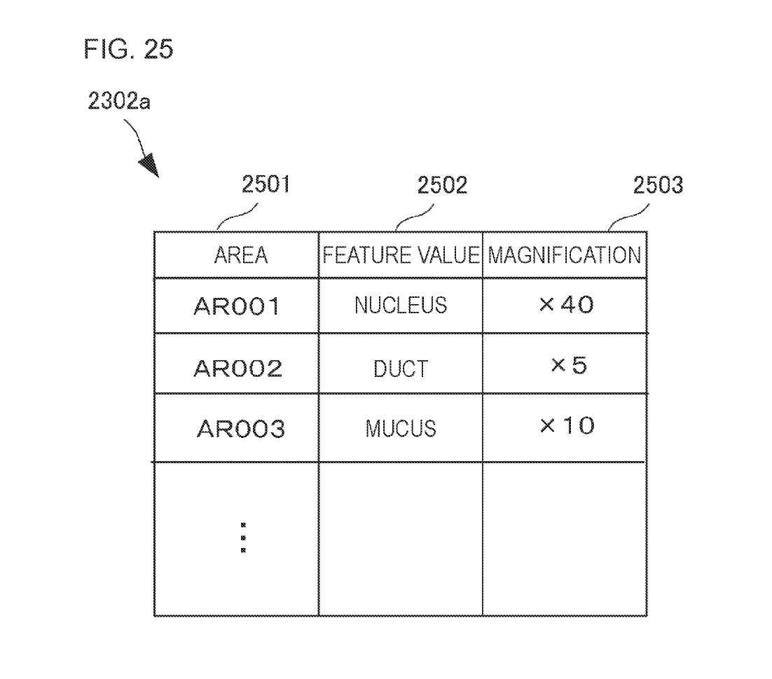

FIG. 25 is a diagram showing a configuration of the magnification selection table 2302a according to the present exemplary embodiment.

In the magnification selection table 2302a, a feature value 2502 obtained from the area generation unit 216 corresponding to an area 2501 that is specified by the pathologist 240, and a magnification 2503 are stored. In addition, although not shown, information is prepared in association with the feature value and the magnification in advance in the magnification selection unit 2302. The information may be stored in another DB. The magnification selection unit 2302 can obtain a magnification associated with the feature value 2502 which is obtained from the area generation unit 216 corresponding to the area 2501 specified by the pathologist 240, using the information. In the example of FIG. 25, as a suitable magnification of the feature value, the magnification ratio of 40 times is selected for the nucleus area, the magnification ratio of five times is selected for the gland tube area, and the magnification ratio of ten times is selected for the mucus area.

### (Expanding Transmission Data)

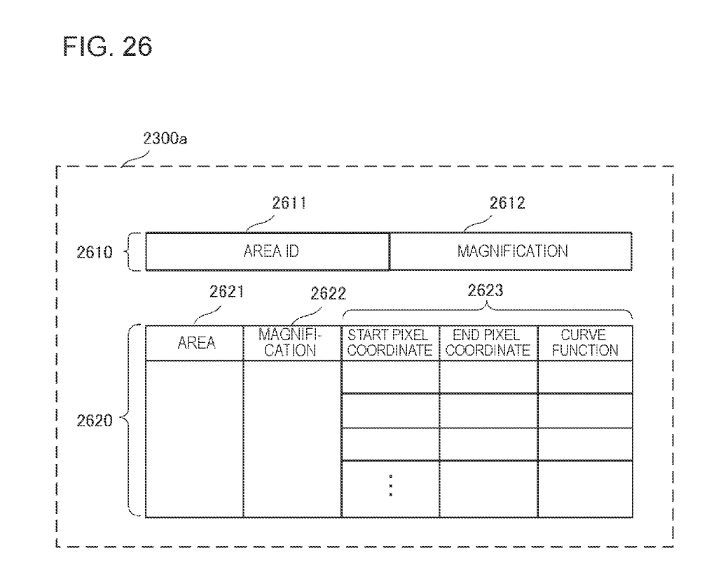

FIG. 26 is a diagram showing the configuration of the expanding transmission data 2300a according to the present exemplary embodiment.

In FIG. 26, 2610 is expanding transmission data which can reduce the amount of information the most, and includes only an area ID 2611 and a magnification 2612. In FIG. 26, 2620 is the second-best expanding transmission data including area information, and a magnification 2622 and outline vectors 2623 of an area are stored in association with the area 2621. According to the expanding transmission data 2300a indicated by 2620, it is possible to use the outline vectors 2623 of an area, and thus the expanding process is simplified.

### <<Processing Procedure of an Information Processing Apparatus>>

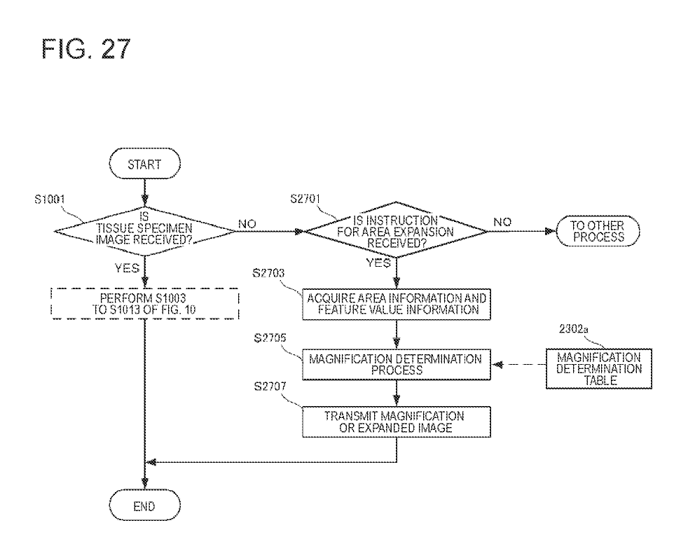

FIG. 27 is a flowchart showing a processing procedure of the information processing apparatus 2310 according to the present exemplary embodiment. In addition, in FIG. 27, the same steps as in FIG. 10 of the second exemplary embodiment are denoted by the same step numbers, and thus the description thereof is not repeated.

In FIG. 27, a branch of step S2701 is newly added. In step S2701, the information processing apparatus 2310 determines whether an instruction for area expansion has been received from the communication terminal 230.

If it is determined that the instruction for area expansion is received, the information processing apparatus 2310 proceeds to step S2703 and acquires area information from the received expanded area information. Then, the information processing apparatus 2310 acquires feature value information from the area generation unit 216, using the acquired area information. Then, in step S2705, the information processing apparatus 2310 selects a magnification according to the feature value information corresponding to the acquired area information, using the magnification selection table 2302a. Then, in step S2707, the information processing apparatus 2310 transmits only the magnification or the expanded area image to the communication terminal 230.

### Seventh Exemplary Embodiment

Next, an information processing system according to the seventh exemplary embodiment of the present invention will be described. The information processing system according to the present exemplary embodiment is different from that in the second exemplary embodiment in that the same image is allocated and displayed for the feature value and level common to a plurality of tissue specimen images.

According to the present exemplary embodiment, it is possible to determine at a glance the feature value or the level to which the pathologist has to pay attention over a plurality of tissue specimen images.

In addition, only the characteristic configuration of the present exemplary embodiment is described, other configurations and operations are the same as in the second exemplary embodiment, and thus the detailed description is not repeated.

(Screen in which an Overlay Image is Superimposed on a Plurality of Tissue Specimen Images)

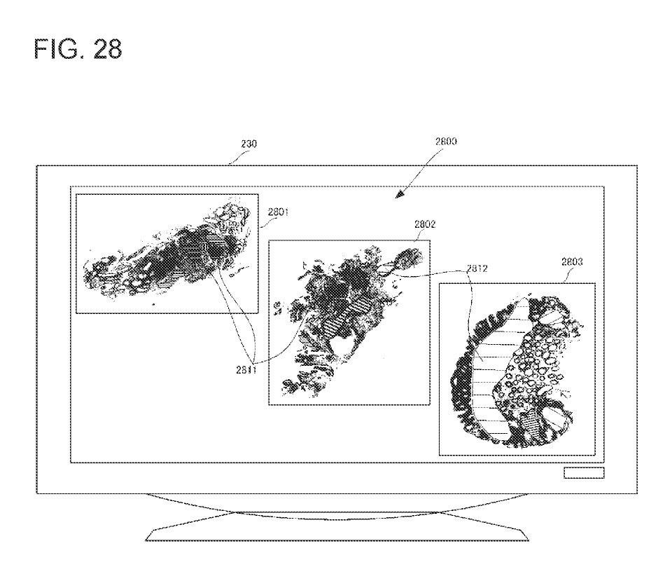

FIG. 28 is a drawing showing a screen 2800 in which an overlay image is superimposed on a plurality of tissue specimen images according to the present exemplary embodiment, in the communication terminal 230. FIG. 28 shows three tissue specimen images, but the number of the tissue specimen images is not limited. However, if the number of the tissue specimen images is large, each tissue specimen image is displayed at a small size and it is difficult to determine an area, such that for example, it is desirable to roll and display the next tissue specimen image.

In FIG. 28, 2801 to 2803 are three tissue specimen images. The overlay image by the common allocation image is superimposed on each tissue specimen image. For example, 2811 and 2812 each shows the same level of the same feature value. In addition, the overlay image may be common to three tissue specimen images or separate for each tissue specimen image.

### (Area Information)

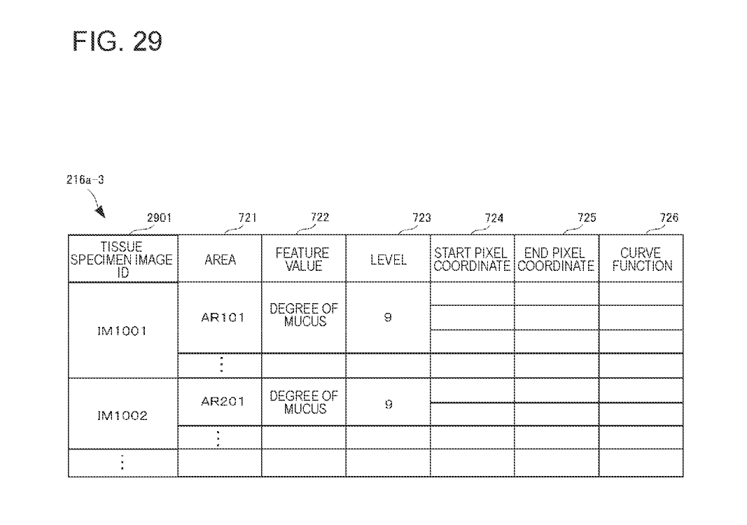

FIG. 29 is a drawing showing area information 216a-3 according to the present exemplary embodiment. The area information 216a-3 has a configuration corresponding to another example 216a-2 of the area information of the second exemplary embodiment. In addition, the same data items as in another example 216a-2 of the area information of the second exemplary embodiment are denoted by the same reference numerals, and thus the description thereof is not repeated.

The area information 216a-3 has the same configuration as in FIG. 7B in the area 721 and the subsequent data, except that a tissue specimen image ID 2901 is added at the beginning thereof. FIG. 29 shows an example in which the area (AR101) of a tissue specimen image ID (IM1001) and the area (AR201) of a tissue specimen image ID (IM1002) have the same feature value (degree of mucus) of level “9”.

### (Overlay Image Information)

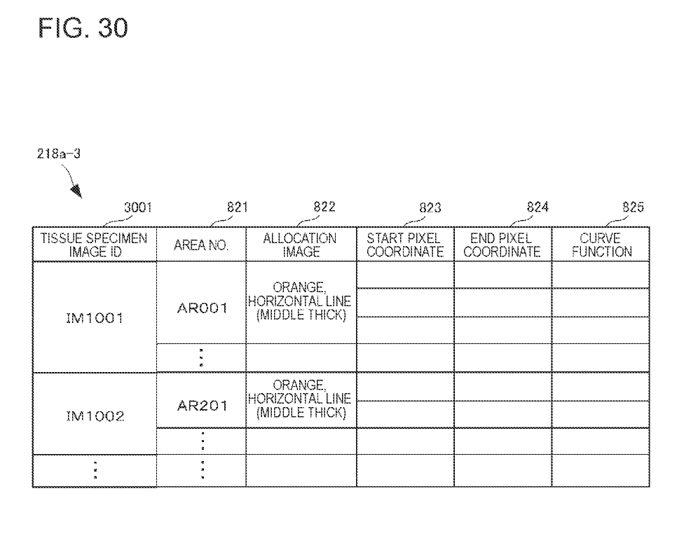

FIG. 30 is a drawing showing overlay image information 218a-3 according to the present exemplary embodiment. The overlay image information 218a-3 has a configuration corresponding to another example 218a-2 of the overlay image information of the second exemplary embodiment. In addition, the same data items as in another example 218a-2 of the overlay image information of the second exemplary embodiment are denoted by the same reference numerals, and thus the description thereof is not repeated.

The overlay image information 218a-3 has the same configuration as FIG. 8B in the area 821 and the subsequent data, except that a tissue specimen image ID 3001 is added at the beginning thereof. In FIG. 30, since the area (AR101) of a tissue specimen image ID (IM1001) and the area (AR201) of a tissue specimen image ID (IM1002) have the same feature value (degree of mucus) of level “9”, the same allocation image (orange, horizontal line of medium thickness) is allocated. Therefore, in the present exemplary embodiment, it is possible to present a common criterion available to a plurality of tissue specimen images, and the pathologist 240 can determine which area over the plurality of tissue specimen images according to the criterion is subjected to detailed diagnosis.

### Other Exemplary Embodiments

Hitherto, the exemplary embodiments of the present invention have been described in detail, but the system or the device in which the different features included in each exemplary embodiment are combined in any way also falls within the scope of the present invention.

Further, the present invention may be applied to a system configured from a plurality of devices, or may be applied to a single device. Further, the present invention is applicable when a control program for realizing functions of the exemplary embodiment is supplied to the device or the system directly or remotely. Accordingly, in order for a computer to perform the functions of the present invention, a control program installed in the computer, a medium storing the control program, or a World Wide Web (WWW) server from which the control program is downloaded is also included in the scope of the present invention.

This application claims priority based on Japanese Patent Application No. 2011-179094 filed on Aug. 18, 2011, which is incorporated herein in its entirety by disclosure.

1. An information processing apparatus which supports diagnosis based on a tissue specimen image obtained by capturing body tissues, comprising:
an area generation unit that divides at least one feature value of the tissue specimen image into a plurality of levels based on a magnitude of the feature value, and generates an area on the tissue specimen image belonging to each level; and
an overlay image generation unit that associates the area of each level which is generated by the area generation unit with an image which is processed to have the same shape and the same positional relation as the area and in which a magnitude relation of the feature value is identifiable, and generates an overlay image.

2. The information processing apparatus according to claim 1,
wherein the feature value includes a degree of differentiation representing a degree of differentiation of cancer cells, a grade that is an evaluation of a histopathological grade of cancer cells, a nuclear grade which is an evaluation by a size or a shape of a cell nucleus, a structural grade representing a degree of gland tube formation, a number or percentage of occurrences of nuclear fission of the cell nucleus, a degree of mucus secreted from a mucosa and a gland, and a possibility of a signet-ring cell cancer, and any combination thereof.

3. The information processing apparatus according to claim 1,
wherein the overlay image generation unit allocates to the area, an image having different hues of color, an image having different brightnesses of color, or an image having a pattern with different degrees of attention, as the image in which the magnitude relation of the feature value is identifiable.

4. The information processing apparatus according to claim 3,
wherein when there are a plurality of feature values, the overlay image generation unit allocates different colors or different patterns to the different feature values.

5. The information processing apparatus according to claim 4,
wherein the area generation unit divides each of three feature values of the tissue specimen image into a plurality of levels based on the magnitude of the feature value, and generates an area on the tissue specimen image belonging to each level, and
wherein the overlay image generation unit allocates one of three primary colors of light to each of the three feature values, and generates three overlay images.

6. The information processing apparatus according to claim 5,
wherein the three feature values are a nuclear grade of a cell nucleus, a structural grade of a gland tube, and a degree of secreted mucus.

7. The information processing apparatus according to claim 1, further comprising:
a tissue specimen image reception unit that receives the tissue specimen image through a network; and
an overlay image transmission unit that transmits the overlay image generated by the overlay image generation unit through the network.

8. The information processing apparatus according to claim 1, further comprising:
a feature value selection unit that selects at least one feature value,
wherein the area generation unit divides the selected feature value into a plurality of levels based on the magnitude of the feature value, and generates an area on the tissue specimen image belonging to each level, and
wherein the overlay image generation unit generates overlay images respectively corresponding to the selected feature values.

9. The information processing apparatus according to claim 8, further comprising:
a specific information reception unit that receives specific information for specifying the tissue specimen image,
wherein the feature value selection unit selects at least one feature value, based on the received specific information.

10. The information processing apparatus according to claim 1, further comprising:
an image selection unit that selects an allocation image including an image in which the magnitude of the feature value is identifiable,
wherein the overlay image generation unit allocates the each selected allocation image to the area of each level generated by the area generation unit.

11. The information processing apparatus according to claim 1, further comprising:
a magnification selection unit that selects a magnification of the tissue specimen image according to the feature value of the area, in response to an instruction for the area; and
a magnification transmission unit that transmits the magnification selected by the magnification selection unit in association with the area through the network.

12. The information processing apparatus according to claim 1,
wherein the tissue specimen image includes a plurality of tissue specimen images, and
the overlay image generation unit allocates the same image to an area having the same level of the same feature value.

13. A control method of an information processing apparatus which supports diagnosis based on a tissue specimen image obtained by capturing body tissues, comprising:
an area generation step of dividing at least one feature value of the tissue specimen image into a plurality of levels based on a magnitude of the feature value, and generating an area on the tissue specimen image belonging to each level; and
an overlay image generation step of associating the area of each level which is generated in the area generation step with an image which is processed to have the same shape and the same positional relation as the area and in which a magnitude relation of the feature value is identifiable, and generating an overlay image.

14. A non-transitory computer readable medium storing a control program of an information processing apparatus which supports diagnosis based on a tissue specimen image obtained by capturing body tissues, the program causing a computer to implement:
an area generation step of dividing at least one feature value of the tissue specimen image into a plurality of levels based on a magnitude of the feature value, and generating an area on the tissue specimen image belonging to each level; and
an overlay image generation step of associating the area of each level which is generated in the area generation step with an image which is processed to have the same shape and the same positional relation as the area and in which a magnitude relation of the feature value is identifiable, and generating an overlay image.

15. An information processing system which supports diagnosis based on a tissue specimen image obtained by capturing body tissues, comprising:
an input unit that inputs the captured tissue specimen image;
an area generation unit that divides at least one feature value of the tissue specimen image into a plurality of levels based on a magnitude of the feature value, and generates an area on the tissue specimen image belonging to each level;
an overlay image generation unit that associates the area of each level which is generated by the area generation unit with an image which is processed to have the same shape and the same positional relation as the area and in which a magnitude relation of the feature value is identifiable, and generates an overlay image; and
a superimposing and displaying unit that superimposes the overlay image generated by the overlay image generation unit on the tissue specimen image, and displays the superimposed image.

16. An information processing method which supports diagnosis based on a tissue specimen image obtained by capturing body tissues, comprising:
an input step of inputting the captured tissue specimen image;
an area generation step of dividing at least one feature value of the tissue specimen image into a plurality of levels based on a magnitude of the feature value, and generating an area on the tissue specimen image belonging to each level;
an overlay image generation step of associating the area of each level which is generated in the area generation step with an image which is processed to have the same shape and the same positional relation as the area and in which a magnitude relation of the feature value is identifiable, and generating an overlay image; and
a superimposing and displaying step of superimposing the overlay image generated in the overlay image generation step on the tissue specimen image, and displaying the superimposed image.
